(19) United States
(12) Patent Application Publication
(10) Pub. No.: US 2025/0001066 A1
(43) Pub. Date: Jan. 2, 2025

(54) SYSTEMS, METHODS, AND APPARATUSES FOR FLUID LEVEL DETECTION IN FLUID STORAGE CANISTERS

(71) Applicant: KCI Manufacturing Unlimited Company, Westmeath (IE)

(72) Inventors: Benjamin A. PRATT, Poole (GB); James K. SEDDON, Wimborne (GB); Michael B. BEASLEY, Wimborne (GB)

(21) Appl. No.: 18/708,724

(22) PCT Filed: Oct. 27, 2022
(86) PCT No.: PCT/IB2022/060354

§ 371 Date: May 9, 2024

Related U.S. Application Data
(60) Provisional application No. 63/282,324, filed on Nov. 23, 2021.

(51) Int. Cl. A61M 1/00

(52) U.S. Cl. CPC A61M 1/982; A61M 1/60; A61M 1/96; A61M 2205/103; A61M 2205/3306; A61M 2205/3379; A61M 2205/502; A61M 2205/583

(57) ABSTRACT

Apparatuses, systems, and methods for determining a value of a parameter associated with a canister are described. The system includes a therapy unit, a canister, and at least one sensor. The therapy unit includes at least one wall with an interior surface and an exterior surface and a controller. The canister includes a canister body and a lid configured to be coupled to the canister body to form a sealed interior. The lid is further configured to be coupled to the exterior surface of the at least one wall of the therapy unit. The at least one sensor is communicatively coupled to the controller and is configured to generate a signal indicative of a parameter associated with the canister. The controller is configured to receive the signal from the at least one sensor and to determine a value of the parameter associated with the canister.

### CROSS-REFERENCE TO RELATED APPLICATIONS

This application is a U.S. National Stage Entry of PCT/IB2022/060354, filed Oct. 27, 2022, which claims the benefit, under 35 U.S.C. § 119 (e), of U.S. Provisional Application No. 63/282,324, filed on Nov. 23, 2021, each of which is incorporated herein by reference in its entirety.

### TECHNICAL FIELD

The invention set forth in the appended claims relates generally to tissue treatment systems and more particularly, but without limitation, to systems, methods, and apparatuses for improved fluid level detection in fluid storage canisters.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for improved fluid level detection in fluid storage canisters in a negative-pressure therapy environment are set forth in the appended claims. Illustrative embodiments are also provided to enable a person skilled in the art to make and use the claimed subject matter.

For example, in some embodiments, a system for fluid level detection can be described. The system can include a therapy unit, a canister, and at least one sensor. The therapy unit can include at least one wall having an interior surface and an exterior surface and a controller. The canister can include a canister body and a lid configured to be coupled to the canister body to form a sealed interior. The lid can be further configured to be coupled to the exterior surface of the at least one wall of the therapy unit. The at least one sensor can be communicatively coupled to the controller and can be configured to generate a signal indicative of a parameter associated with the canister. The controller can be configured to receive the signal from the at least one sensor and to determine a value of the parameter associated with the canister. In some examples, the parameter can be an amount of fluid stored in the sealed interior of the canister.

In some example embodiments, the at least one sensor can be coupled to the interior surface of the at least one wall. A probe of the at least one sensor can be directed towards the exterior surface of the at least one wall. In some embodiments, the at least one wall of the therapy unit can include at least one cavity. The lid may include at least one cavity that is configured to align with the at least one cavity of the at least one wall of the therapy unit when the canister is coupled to the therapy unit. The at least one sensor can be disposed proximate to the at least one cavity of the at least one wall of the therapy unit. In other example embodiments, the at least one sensor can protrude from the exterior surface of the at least one wall of the therapy unit or can be coupled to the exterior surface of the at least one wall of the therapy unit.

In other example embodiments, the lid of the canister can comprise at least one protrusion that can be configured to align with at least one recess on the at least one wall of the therapy unit. The at least one sensor can be proximate to the interior surface of the at least one wall of the therapy unit. In some examples, a probe of the at least one sensor can be directed towards the at least one recess on the at least one wall of the therapy unit. The parameter associated with the canister can be an amount of fluid in the at least one protrusion of the lid of the canister.

In some example embodiments, the system can include a user interface configured to be communicatively coupled to the controller. The user interface can also output the value of the parameter associated with the canister. In other example embodiments, the system can include an orientation detector. The orientation detector can be configured to generate a signal indicative of an orientation of the canister. The orientation detector can further be configured to be communicatively coupled to the controller. The controller can be configured to receive the signal from the orientation detector to determine the value of the parameter associated with the canister. In still other example embodiments, the therapy unit can further include an airflow device that can be configured to be actuated by the controller in response to the parameter associated with the controller.

A method of determining a value of a parameter of a canister is also described herein. In some embodiments, a therapy unit, a canister, and a plurality of sensors can be provided. The therapy unit can include at least one wall with an interior surface and an exterior surface and a controller. The canister can include a canister body and a lid configured to attach to the canister body to form a sealed interior. The lid can be further configured to couple to the exterior surface of the at least one wall of the therapy unit. Each sensor of the plurality of sensors can be configured to be communicatively coupled to the controller and to generate a signal indicative of a parameter associated with the canister. Each sensor can generate a signal indicative of the parameter associated with the canister. The controller can receive each signal from each sensor of the plurality of sensors. A value of the parameter associated with the canister can be determined in response to the signal from each sensor of the plurality of sensors.

In some example embodiments, a user interface can be communicatively coupled to the controller. The user interface can receive the value of the parameter associated with the canister from the controller and can output the value of the parameter associated with the controller. In other example embodiments, the controller can store historical data associated with the canister. The value of the parameter associated with the canister can be determined in response to the historical data associated with the parameter associated with the canister. In still other example embodiments, an orientation detector can be provided. The orientation detector can be configured to generate an orientation signal indicative of an orientation of the canister. The orientation detector can be communicatively coupled to the controller. The controller can receive the orientation signal from the orientation detector and can determine the value of the parameter associated with the canister in response to the orientation signal.

In some embodiments, the parameter can be an amount of fluid in the sealed interior of the canister. To generate the signal with each sensor of the plurality of sensors, a probe of each sensor can be directed towards the sealed interior of the canister. Each sensor may sense fluid in a sensing path of the probe of the sensor. The signal from each sensor of the plurality of sensors may be set to a value indicative of a sensed fluid if the sensor senses fluid in the sensing path of the probe or may be set to a value indicative of a lack of fluid if the sensor does not sense fluid in the sensing path of the probe. Each signal may be sent from each sensor of the plurality of sensors to the controller.

In other example embodiments, to generate the signal with each sensor of the plurality of sensors, the plurality of sensors can be disposed proximate to the interior surface of the at least one wall of the therapy unit. A probe of each sensor can be directed towards a protrusion of a plurality of protrusions of the lid of the canister. Each protrusion of the plurality of protrusions can be configured to be received by a recess of a plurality of recesses of the at least one wall of the canister. Each sensor can sense fluid in a corresponding protrusion. The signal from each sensor of the plurality of sensors may be set to a value indicative of a sensed fluid if the sensor senses fluid in the corresponding protrusion or may be set to a value indicative of a lack of fluid if the sensor does not sense fluid in the corresponding protrusion. Each signal may be sent from each sensor of the plurality of sensors to the controller.

A canister is also described herein. In some example embodiments, the canister can include a canister body, a lid, and at least one sensor. The lid can be configured to attach to the canister body to form a sealed interior. The at least one sensor can include a probe that can be directed towards the sealed interior of the canister. The at least one sensor can be configured to generate a signal indicative of a parameter associated with the canister. In some embodiments, the at least one sensor can be a sensor array. The at least one sensor can be integrated into the lid of the canister. In other embodiments, the at least one sensor can be integrated into the canister body.

In some example embodiments, the canister can include an orientation detector. The orientation detector can be configured to generate a signal indicative of an orientation of the canister. In other example embodiments, the canister can include an airflow device. The airflow device can be configured to generate a fluid flow through the sealed interior of the canister when the airflow device is actuated. The airflow device can be actuated when a value of the parameter associated with the canister is equal to a predetermined value.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment in accordance with this specification;

FIG. 2 is a perspective view of an illustrative example embodiment of a therapy unit and a canister of the therapy system of FIG. 1;

FIG. 3 is an exploded view of the therapy unit and the canister of FIG. 2;

FIG. 4 is a perspective view of an illustrative example embodiment of the therapy unit of FIG. 2;

FIG. 5A is a right-side view of an illustrative example embodiment of the therapy unit and the canister of FIG. 2 in a first orientation with a first amount of fluid in a sealed interior of the canister;

FIG. 5B is a right-side view of an illustrative example embodiment of the canister of FIG. 2 in the first orientation with a second amount of fluid in the sealed interior of the canister;

FIG. 5C is a right-side view of an illustrative example embodiment of the canister of FIG. 2 in the first orientation with a third amount of fluid in the sealed interior of the canister;

FIG. 6A is a right-side view of an illustrative example embodiment of the canister of FIG. 2 in a second orientation with a first amount of fluid in the sealed interior of the canister;

FIG. 6B is a right-side view of an illustrative example embodiment of the canister of FIG. 2 in the second orientation with a second amount of fluid in the sealed interior of the canister;

FIG. 6C is a right-side view of an illustrative example embodiment of the canister of FIG. 2 in the second orientation with a third amount of fluid in the sealed interior of the canister;

FIG. 7A is a perspective view of an illustrative example embodiment of the canister of FIG. 2 in a third orientation with a first amount of fluid in the sealed interior of the canister;

FIG. 7B is a perspective view of an illustrative example embodiment of the canister of FIG. 2 in the third orientation with a second amount of fluid in the sealed interior of the canister;

FIG. 7C is a perspective view of an illustrative example embodiment of the canister of FIG. 2 in the third orientation with a third amount of fluid in the sealed interior of the canister;

FIG. 8A is a perspective view of an illustrative example embodiment of a home screen of a user interface that may be associated with the therapy unit of FIG. 2;

FIG. 8B is a perspective view of another illustrative example embodiment of the home screen of the user interface that may be associated with the therapy unit of FIG. 2;

FIG. 8C is a perspective view of another illustrative example embodiment of the home screen of the user interface that may be associated with the therapy unit of FIG. 2;

FIG. 8D is a perspective view of another illustrative example embodiment of the home screen of the user interface that may be associated with the therapy unit of FIG. 2;

FIG. 8E is a perspective view of another illustrative example embodiment of the home screen of the user interface that may be associated with the therapy unit of FIG. 2;

FIG. 9 is a cut away view of an illustrative example embodiment of the therapy unit and the canister of FIG. 1;

FIG. 10A is a perspective view of an illustrative example embodiment of the therapy unit and the canister of FIG. 1 with a sensor module;

FIG. 10B is an exploded view of the therapy unit, the canister, and the sensor module of FIG. 10A;

FIG. 11 is a perspective view of an illustrative example embodiment of the therapy unit and a sensor array of FIG. 1;

FIG. 12 is a perspective view of an illustrative example embodiment of the therapy unit and another sensor array of FIG. 1;

FIG. 13 is a perspective view of an illustrative example embodiment of the therapy unit and the canister of FIG. 1 with another example of the sensor array integrated into the canister;

FIG. 14A is a perspective view with a partial cut-away view of an illustrative example embodiment of the therapy unit and the canister of FIG. 1; and

FIG. 14B is an exploded partial cut-away view of an illustrative example embodiment of the therapy unit and canister of FIG. 14A.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientations assume a frame of reference consistent with or relative to a patient in a position to receive treatment. However, as should be recognized by those skilled in the art, this frame of reference is merely a descriptive expedient rather than a strict prescription.

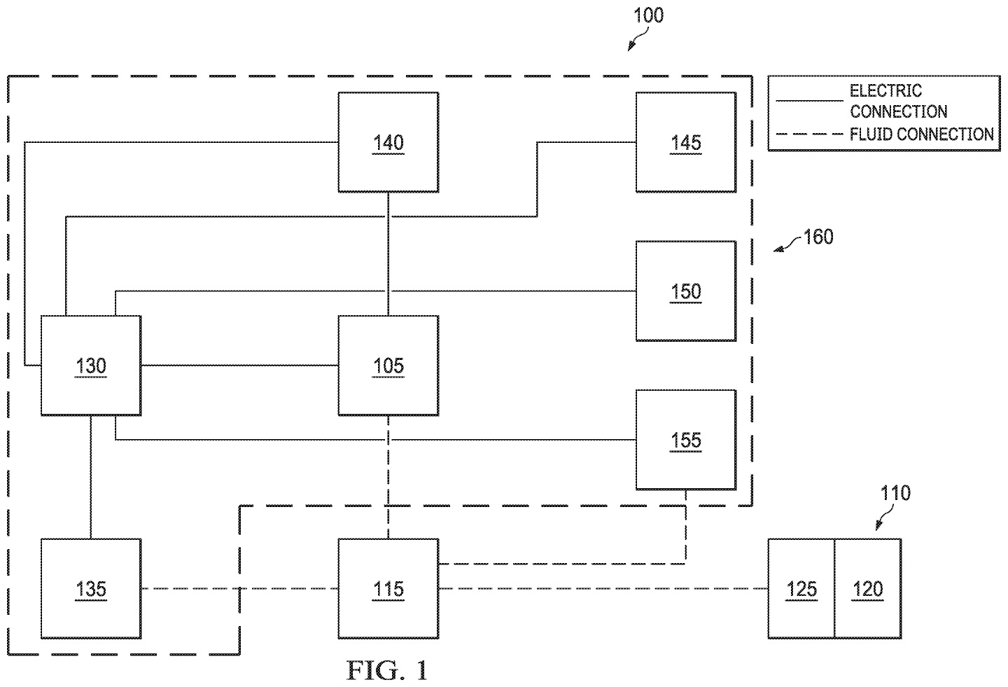

FIG. 1 is a simplified functional block diagram of an example embodiment of a therapy system 100 that can provide negative-pressure therapy to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a canister 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135, a second sensor 140, and a third sensor, such as a sensor array 145 coupled to the controller 130. The sensor array 145 may include one or more sensors that may each be communicatively coupled to the controller 130.

In some embodiments, the therapy system 100 may also include a fan, a fluid flow device, or an airflow device 150 that may be communicatively coupled to the controller 130. The therapy system 100 may also include an accelerometer, a gyroscope, or an orientation detector 155. The orientation detector 155 may be configured to sense an orientation of the canister 115 and may be communicatively coupled to the controller 130. The airflow device 150 and the orientation detector 155 may be configured to provide a signal indicative of a status of the airflow device 150 and the orientation detector 155, respectively, to the controller 130 similar to the first sensor 135, the second sensor 140, and the sensor array 145.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, and other components into a therapy unit 160. As shown in FIG. 1, the negative-pressure source 105, the controller 130, the first sensor 135, the second sensor 140, the sensor array 145, the airflow device 150, and the orientation detector 155 may all be included in the therapy unit 160. In other embodiments, the sensor array 145, the airflow device 150, and/or the orientation detector 155 may be located outside of the therapy unit 160 and may be located within the canister 115 or in a different location in the therapy system 100.

In general, components of the therapy system 100 may be coupled directly or indirectly. For example, the negative-pressure source 105 may be directly coupled to the canister 115 and may be indirectly coupled to the dressing 110 through the canister 115. Coupling may include fluid, mechanical, thermal, electrical, communicative (such as a wireless connection), or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between-5 mm Hg (−667 Pa) and −500 mm Hg (−66.7 kPa). Common therapeutic ranges are between-50 mm Hg (−6.7 kPa) and −300 mm Hg (−39.9 kPa).

The canister 115 is representative of a container, canister, pouch, or other storage component, which can be used to manage exudates and other fluids withdrawn from a tissue site. In many environments, a rigid canister may be preferred or required for collecting, storing, and disposing of fluids. In other environments, fluids may be properly disposed of without rigid canister storage, and a re-usable canister could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, the power applied to the airflow device 150, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135, the second sensor 140, and the sensor array 145, can be any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. The sensor array 145 may be configured to measure a parameter associated with the canister 115 such as a fluid level stored in the canister 115 or a color of fluid stored in the canister 115. Preferably, the signals from the first sensor 135, the second sensor 140, and the sensor array 145 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inspire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in canister 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

The controller 130 may also be configured to receive and process data from the sensor array 145. The controller 130 may be configured to determine a value of a parameter associated with the canister 115 from the data received from the sensor array 145. For example, the controller 130 may be configured to receive and process data from the sensor array 145 to determine the amount of fluid stored in the canister 115. In some embodiments, the controller 130 may also be configured to receive data from the orientation detector 155 and to process the data to determine the value of the parameter associated with the canister 115.

Negative-pressure therapy has been repeatedly shown to be effective in the treatment of difficult to heal wounds. Manufacturers are designing current negative-pressure therapy systems to enable patients to be ambulatory. To help patients to feel comfortable being ambulatory, current negative-pressure therapy systems are also being designed to include opaque canisters to provide increased privacy to patients. Opaque canisters provide increased privacy but also make it difficult for health care providers to determine an accurate fill level of the canister. Without having an accurate fill level of an ambulatory canister, the canister may be replaced before it is full which may increase costs and reduce patient satisfaction with the negative-pressure therapy system. A negative-pressure therapy system capable of monitoring the fill level of a canister in any orientation and providing real time updates to patients and health care providers of the fill level of the canister is a long sought but unsolved solution in the art.

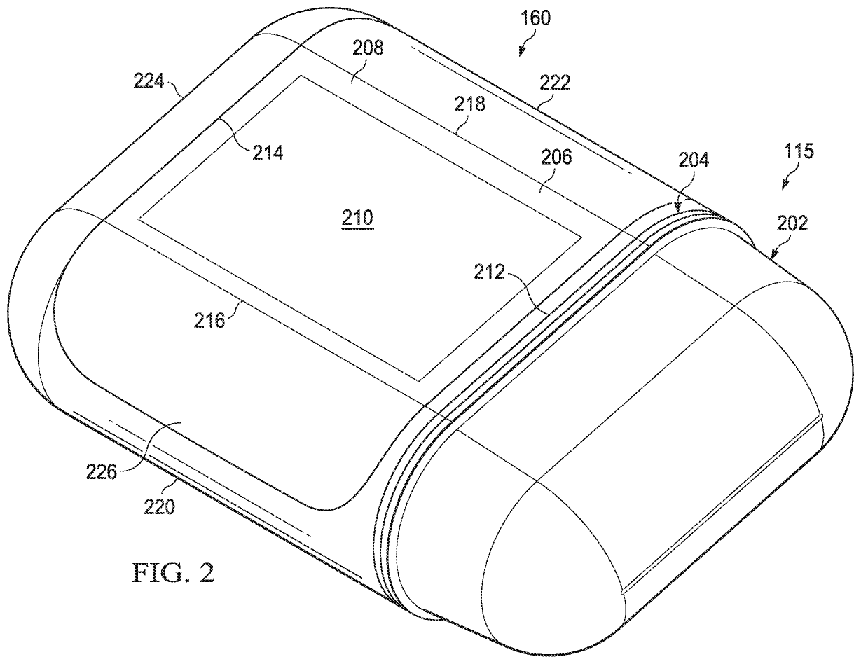

FIG. 2 is a perspective view of an example embodiment of the therapy unit 160 and the canister 115 of FIG. 1. In some embodiments, the negative-pressure source 105, the controller 130, the sensor array 145, the orientation detector 155, and the airflow device 150 may be disposed within the therapy unit 160. In other embodiments, the sensor array 145, the orientation detector 155, and the airflow device 150 may be disposed within the canister 115 or in a different location in the therapy system 100.

The canister 115 may include a canister body 202 and a lid 204. The lid 204 may be coupled to the canister body 202 to form a sealed interior in the canister 115 between the canister body 202 and the lid 204. The lid 204 may be coupled to the canister body 202 at one or more attachment points, by one or more adhesives, or other suitable methods of creating the sealed interior. In some embodiments, the canister body 202 may be substantially opaque.

The canister 115 may comprise a type of material having sufficient rigidity and structural integrity to withstand the negative pressure required for negative-pressure treatment and to contain fluid therein. Some exemplary materials of the canister 115 are plastics, polymers, thermoplastics, metals, metal alloys, composition material, fiber-type materials, and other similar materials. The plastics described herein may be a substance or structure capable of being shaped or molded with or without the application of heat, a high polymer, usually synthetic, combined with other ingredients such as curatives, fillers, reinforcing agents, plasticizers, etc. Plastics can be formed or molded under heat and pressure in its raw state and machined to high dimensional accuracy, trimmed and finished in its hardened state. The thermoplastic type can be resoftened to its original condition by heat. In addition, the plastics may mean engineered plastics such as those that are capable of sustaining high levels of stress and are machinable and dimensionally stable. Some exemplary plastics are nylon, acetyls, polycarbonates, ABS resins, PPO/styrene, ISOPLAST 2530, TURLUX HS 2822, and polybutylene terephthalate. The thermoplastics described herein may be high polymers that soften when exposed to heat and return to their original condition when cooled to room temperature.

The therapy unit 160 may have a first wall 206 that may have an exterior surface 208 and an interior surface opposite the exterior surface 208. In some embodiments, there may be a user interface 210 coupled to the exterior surface 208 of the first wall 206. The user interface 210 may be communicatively coupled to the controller 130, which may be located within the therapy unit 160. The first wall 206 may have a first edge 212, a second edge 214 opposite the first edge 212, a third edge 216 configured to connect the first edge 212 and the second edge 214, and a fourth edge 218 opposite the third edge 216.

There may be a second wall opposite the first wall 206 of the therapy unit 160. The third edge 216 of the first wall 206 may be coupled to the second wall by a first connection 220 of the therapy unit 160. The first connection 220 may be a curved or beveled wall that extends from the third edge 216 of the first wall 206 to the second wall. In other embodiments, the first connection 220 may be a different shape such as a flat wall similar to the first wall 206. There may be a second connection 222 opposite the first connection 220. The second connection 222 may couple the fourth edge 218 of the first wall 206 to the second wall. The therapy unit 160 may also have a base 224 that couples the second edge 214 of the first wall 206 to the second wall. The base 224 may be similar in both size and shape to the first connection 220 and the second connection 222. The therapy unit 160 may further include a canister connection wall that is configured to couple to the lid 204 of the canister 115.

In some embodiments, the therapy unit 160 may have an area 226 that covers most of the first wall 206 and extends over approximately half of the surface area of the first connection 220 and the second connection 222. The area 226 may be a panel that may couple to the therapy unit 160 or may be formed integrally with the therapy unit 160. The area 226 may allow easier integration of the user interface 210 with the therapy unit 160. In other embodiments, the area 226 may not be included in the therapy unit 160.

In some embodiments, the therapy unit 160 may be formed from materials similar to those described above with respect to the canister 115.

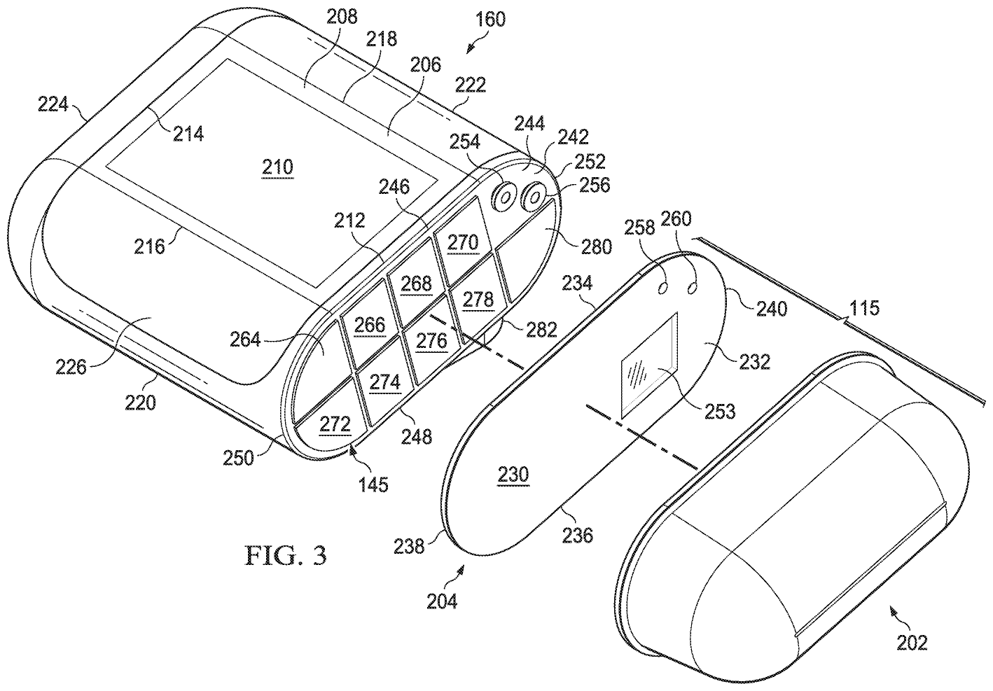

FIG. 3 is an exploded view of the therapy unit 160 and the canister 115 of FIG. 2. The lid 204 of the canister 115 may have an interior surface or a first surface 230 and an exterior surface or a second surface opposite the first surface 230. The first surface 230 of the lid 204 may have a periphery 232 that may be configured to couple to the canister body 202. When the lid 204 is coupled to the canister body 202, the sealed interior of the canister 115 may be formed. In some embodiments, the lid 204 may be in the shape of a stadium. The lid may have a first edge 234, a second edge 236 opposite the first edge 234, a third edge 238 in the shape of a semicircle that connects the first edge 234 and the second edge 236, and a fourth edge 240 in the shape of a semicircle opposite the third edge 238 that also connects the first edge 234 and the second edge 236. In other embodiments, the lid 204 may be circular, rectangular, ovular, or another shape that may couple to the canister body 202 to create the scaled interior.

The second surface of the lid 204 of the canister 115 may be removably coupled to an exterior surface 242 of the canister connection wall or a third wall 244 of the therapy unit 160. The third wall 244 may be substantially similar in size and shape to the lid 204 of the canister 115. The third wall 244 may have a first edge 246 and a second edge 248 opposite the first edge 246. The third wall 244 may have a third edge 250 in the shape of a semicircle that connects the first edge 246 to the second edge 248. There may be a fourth edge 252 opposite the third edge 250 also in the shape of a semicircle that connects the first edge 246 to the second edge 248. In other embodiments, the third wall 244 may be substantially circular, rectangular, ovular, or another shape that may couple to the second surface of the lid 204.

In some embodiments, the lid 204 of the canister 115 may be transparent. A medical professional may disconnect the canister 115 from the therapy unit 160 and observe fluid in the sealed interior of the canister 115 through the lid 204. In other embodiments, the lid 204 of the canister 115 may be opaque to obscure any fluids in the sealed interior from view. In embodiments where both the canister body 202 and the lid 204 are opaque, a viewing window 253 may optionally be included through a portion of the lid 204. In other embodiments, the viewing window 253 may be included through a portion of the canister body 202. The viewing window may allow the medical professional to observe fluids stored in the sealed interior of the canister 115 while allowing a majority of the canister 115 to remain opaque for increased user privacy and comfort.

In some embodiments, the third wall 244 of the therapy unit 160 may include a first connection point 254 and a second connection point 256. The first connection point 254 and the second connection point 256 may be located proximate to the fourth edge 252 and the first edge 246 of the third wall 244. In other embodiments, the first connection point 254 and the second connection point 256 may be located at a different point on the third wall 244. The lid 204 of the canister 115 may include a third connection point 258 and a fourth connection point 260. The third connection point 258 may be aligned with the first connection point 254 when the canister 115 is coupled to the therapy unit 160 and the fourth connection point 260 may be aligned with the second connection point 256 when the canister 115 is coupled to the therapy unit 160. The first connection point 254, the second connection point 256, the third connection point 258, and the fourth connection point 260 may provide communication between the therapy unit 160 and the canister 115. For example, in some embodiments, the first connection point 254 and the third connection point 258 may be adapted to deliver negative pressure to the dressing 110 and the canister 115. The second connection point 256 and the fourth connection point 260 may provide a sensing path to sense the pressure in the dressing 110 and the canister 115.

The sensor array 145 may be disposed on or coupled to the third wall 244 of the therapy unit 160. In some embodiments, the sensor array 145 may be formed integrally with the third wall 244 of the therapy unit 160. In other embodiments, the sensor array 145 may be coupled to the exterior surface 242 of the third wall 244 of the therapy unit 160. The sensor array 145 may be coupled to the exterior surface 242 by a weld, and adhesive, or another suitable method configured to secure the sensor array 145 to the exterior surface 242.

The sensor array 145 may comprise one or more sensors configured to generate a signal indicative of a parameter associated with the canister 115. Each sensor of the sensor array 145 may be communicatively coupled to the controller 130, and the controller 130 may be configured to receive each signal from each sensor of the sensor array 145. The controller 130 may be further configured to determine a value of the parameter associated with the canister 115. In some embodiments, the parameter associated with the canister 115 may be an amount of fluid in the sealed interior of the canister 115. In other embodiments, the parameter associated with the canister 115 may be a color of the fluid in the sealed interior of the canister, or the parameter may be both the amount of fluid and the color of the fluid in the sealed interior of the canister 115.

In some embodiments, the sensor array 145 may comprise a first sensor 264, a second sensor 266, a third sensor 268, a fourth sensor 270, a fifth sensor 272, a sixth sensor 274, a seventh sensor 276, an eighth sensor 278, and a ninth sensor 280. In some embodiments, the first sensor 264, the second sensor 266, the third sensor 268, the fourth sensor 270, the fifth sensor 272, the sixth sensor 274, the seventh sensor 276, the eighth sensor 278, and the ninth sensor 280 may each include a plurality of sensors that are communicatively coupled to the controller 130. In other embodiments, the first sensor 264, the second sensor 266, the third sensor 268, the fourth sensor 270, the fifth sensor 272, the sixth sensor 274, the seventh sensor 276, the eighth sensor 278, and the ninth sensor 280 may each comprise one sensor that may be communicatively coupled to the controller 130. In either configuration, the first sensor 264, the second sensor 266, the third sensor 268, the fourth sensor 270, the fifth sensor 272, the sixth sensor 274, the seventh sensor 276, the eighth sensor 278, and the ninth sensor 280 may each send at least one signal to the controller 130 indicative of a parameter associated with the canister 115. The controller 130 may receive each signal and determine a value of the parameter associated with the canister 115. In some embodiments, the parameter associated with the canister 115 may be an amount of fluid in the scaled interior of the canister 115. In other embodiments, the parameter associated with the canister 115 may be the color of the fluid in the sealed interior of the canister 115. In still other embodiments, the controller 130 may be configured to determine a value of multiple parameters associated with the canister 115 such as the amount of fluid in the scaled interior of the canister 115 and the color of that fluid.

Each sensor of the sensor array 145 may include a probe. Each probe may be directed towards the sealed interior of the canister 115. Each probe may sense the parameter associated with the canister 115. A sensing path may be formed from each probe through the scaled interior of the canister 115. Each sensing path may be perpendicular to the third wall 244 of the therapy unit 160 and may be unique and non-overlapping with every other sensing path. In some embodiments, each sensor of the sensor array 145 may be configured to determine the presence of fluid along the sensing path of the corresponding probe. In other embodiments, each sensor of the sensor array 145 may be configured to determine the presence of fluid along the sensing path of the corresponding probe and if fluid is present, how much of the sensing path within the sealed interior of the canister 115 is covered in fluid. In some embodiments, the accuracy of the value calculated by the controller 130 may be increased if each sensor of the sensor array 145 can determine how much of its sensing path is covered in fluid. This configuration may allow the controller 130 to determine an accurate value of the amount of fluid in the canister 115 even if the canister 115 is in a complex orientation.

In some embodiments, each sensor of the sensor array 145 may be a non-contact capacitive fluid sensor. Each sensor may communicate with the controller 130 to indicate an amount of fluid that is along the sensing path associated with the sensor in the sealed interior of the canister 115. In some embodiments, each sensor of the sensor array 145 may be a DFRobot XKC-Y25-T12V SKU SEN0204 sensor. In other embodiments, the sensor array 145 may be comprised of a plurality of sensors of a different type such as optical sensors or resistive sensors that can provide a value indicative of a level of fluid in the sealed interior of the canister 115. In some embodiments, optical sensors may include an emitter such as an infrared emitter and a receiver such as a phototransistor receiver. Resistive sensors may communicate a different resistance to the controller 130 depending on a fluid level in the canister 115. In other embodiments, each sensor of the sensor array 145 may be another sensor that can communicate with the controller 130 to determine the level of fluid in the sealed interior of the canister 115.

In some embodiments, the therapy unit 160 may include a stand 282. The stand 282 may allow the therapy unit 160 and the canister 115 to be oriented in an optimal orientation when the therapy unit 160 and the canister 115 are at a stationary location. In some embodiments, the stand 282 may also provide an easy way to transport the therapy unit 160.

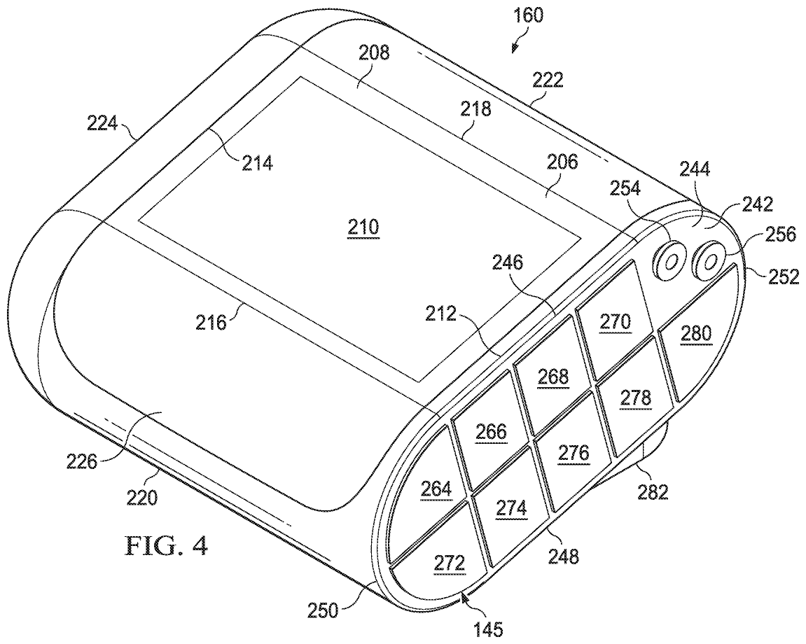

FIG. 4 is a perspective view of an example embodiment of the therapy unit of FIG. 3. The sensor array 145 may either be formed integrally with the third wall 244 of the therapy unit 160 or may be coupled to the exterior surface 242 of the third wall 244 of the therapy unit 160 as described above. The sensor array 145 may include the first sensor 264, the second sensor 266, the third sensor 268, the fourth sensor 270, the fifth sensor 272, the sixth sensor 274, the seventh sensor 276, the eighth sensor 278, and the ninth sensor 280 as described above.

The first sensor 264 may be proximate to the first edge 246 and the third edge 250 of the third wall 244. The first sensor 264 may be in the shape of a sector with a radius approximately equal to the radius of the semicircle defined by the third edge 250. The second sensor 266, the third sensor 268, and the fourth sensor 270 may be in the shape of a square and may be proximate to the first edge 246 of the third wall 244 of the therapy unit 160. The second sensor 266 may be between the first sensor 264 and the third sensor 268. The third sensor 268 may be between the second sensor 266 and the fourth sensor 270. The fifth sensor 272 may be a sector substantially the same size and shape as the first sensor 264. The fifth sensor 272 may be proximate to the third edge 250 and the second edge 248 of the third wall 244. The sixth sensor 274, the seventh sensor 276, and the eighth sensor 278 may be square and may be proximate to the second edge 248 of the third wall 244 of the therapy unit 160. The sixth sensor 274 may be between the fifth sensor 272 and the seventh sensor 276. The seventh sensor 276 may be between the sixth sensor 274 and the eighth sensor 278. The ninth sensor 280 may be a sector substantially the same size and shape as the first sensor 264 and the fifth sensor 272. The ninth sensor 280 may be proximate to the eighth sensor 278, the fourth edge 252, and the second edge 248 of the third wall 244. In other embodiments, each sensor of the sensor array 145 may be a different size or shape but may substantially cover the surface area of the third wall 244 of the therapy unit 160.

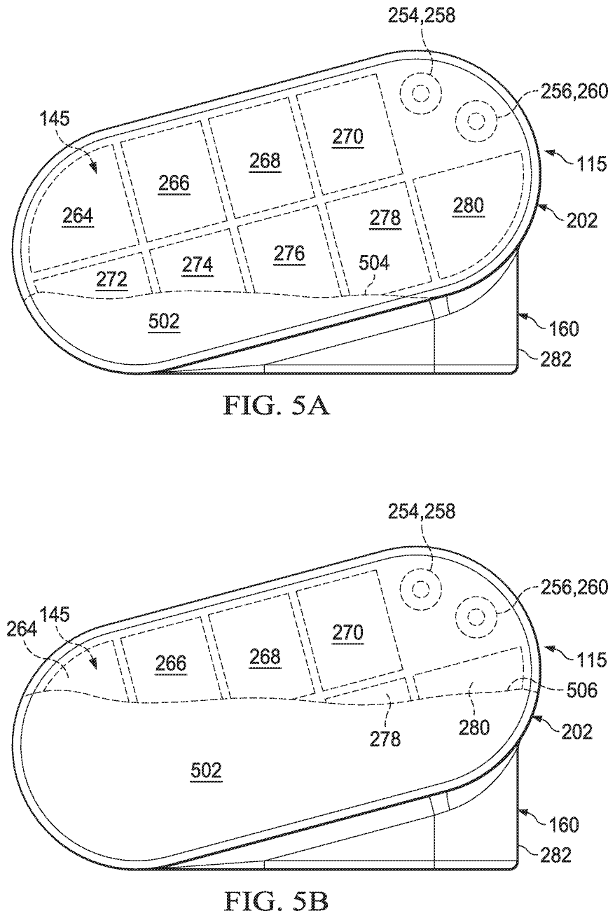

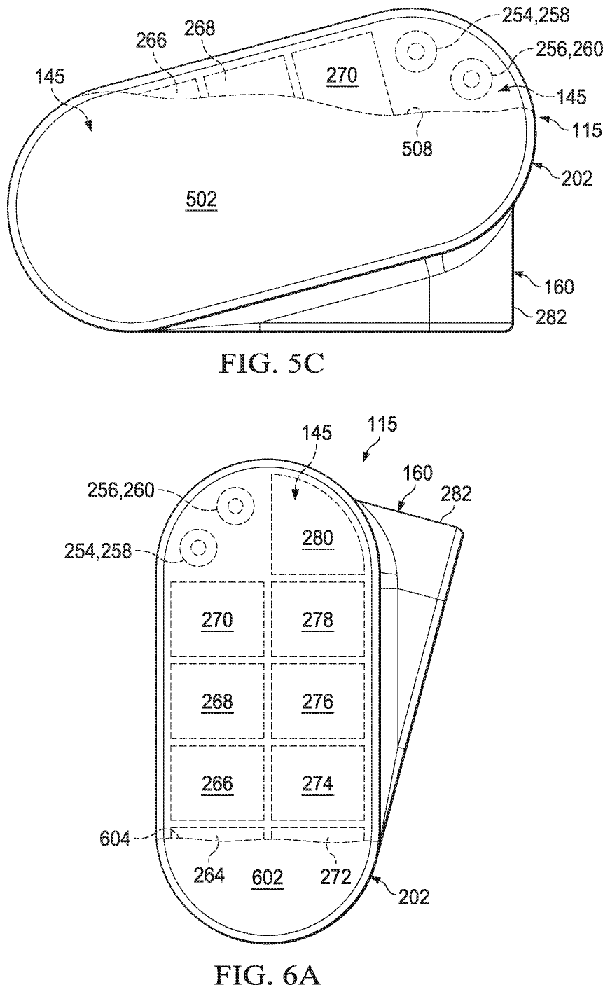

FIGS. 5A-5C are right-side views of example embodiments of the therapy unit 160 and the canister 115 with fluid 502 in the sealed interior of the canister 115. FIGS. 5A-5C show the therapy unit 160 and the canister 115 coupled together and in an orientation where the stand 282 is resting on a horizontal surface. In this orientation, the canister 115 is at an angle relative to the horizontal surface. For example, in some embodiments, the canister 115 may be at an angle of about 20 degrees relative to the horizontal surface. In other embodiments, the canister 115 may be at a different angle relative to the horizontal surface or may be resting on the horizontal surface such that the second edge 236 of the lid 204 is resting on the horizontal surface. In FIGS. 5A-5C, the controller 130 may be located within the therapy unit 160 and may be configured to determine an amount of the fluid 502 stored in the sealed interior of the canister 115.

FIG. 5A shows the canister 115 with a first volume of fluid or a first amount of fluid 504 in the sealed interior. The fifth sensor 272, the sixth sensor 274, the seventh sensor 276, and the eighth sensor 278 may each sense the fluid 502 in the sealed interior of the canister 115. The first sensor 264, the second sensor 266, the third sensor 268, the fourth sensor 270, and the ninth sensor 280 may not sense the fluid 502.

In operation, a probe of the first sensor 264, a probe of the second sensor 266, a probe of the third sensor 268, a probe of the fourth sensor 270, and a probe of the ninth sensor 280 may not sense the presence of the fluid 502 in each of their respective sensing paths. The first sensor 264, the second sensor 266, the third sensor 268, the fourth sensor 270, and the ninth sensor 280 may communicate a signal to the controller 130 indicative of the absence of fluid in each of the sensing paths of the first sensor 264, the second sensor 266, the third sensor 268, the fourth sensor 270, and the ninth sensor 280. A probe of the fifth sensor 272, a probe of the sixth sensor 274, a probe of the seventh sensor 276, and a probe of the eighth sensor 278 may each sense the presence of the fluid 502 in a portion of their respective sensing paths. The fifth sensor 272, the sixth sensor 274, the seventh sensor 276, and the eighth sensor 278 may communicate a signal to the controller 130 indicative of the presence of fluid in their respective sensing paths. In some embodiments, the signal associated with each probe corresponding with a respective sensor may further indicate additional information collected about the first amount of fluid 504 in the sealed interior of the canister 115. The controller 130 may receive the signal from each sensor of the sensor array 145 and determine the first amount of fluid 504 in the sealed interior of the canister 115. For example, the controller 130 may determine that the canister is about 20% full and may communicate this value with the user interface 210 of the therapy unit 160.

FIG. 5B shows the canister 115 with a second volume of fluid or a second amount of fluid 506 in the sealed interior. The first sensor 264, the second sensor 266, the third sensor 268, the fifth sensor 272, the sixth sensor 274, the seventh sensor 276, the eighth sensor 278, and the ninth sensor 280 may each sense the fluid 502 in the sealed interior of the canister 115. The fourth sensor 270 may not sense the fluid 502.

In operation, the probe of the fourth sensor 270 may not sense the presence of the fluid 502 in its sensing path. The fourth sensor 270 may communicate a signal to the controller 130 indicative of the absence of fluid in the sensing path of the fourth sensor 270. The probe of the first sensor 264, the probe of the second sensor 266, the probe of the third sensor 268, the probe of the fifth sensor 272, the probe of the sixth sensor 274, the probe of the seventh sensor 276, the probe of the eighth sensor 278, and the probe of the ninth sensor 280 may each sense the presence of the fluid 502 in a portion of their respective sensing paths. The first sensor 264, the second sensor 266, the third sensor 268, fifth sensor 272, the sixth sensor 274, the seventh sensor 276, the eighth sensor 278, and the ninth sensor 280 may then communicate a signal to the controller 130 indicative of the presence of fluid in their respective sensing paths. In some embodiments, the signal associated with each probe corresponding with a respective sensor may further indicate additional information collected about the second amount of fluid 506 in the sealed interior of the canister 115. The controller 130 may receive the signal from each sensor of the sensor array 145 and determine the second amount of fluid 506 in the sealed interior of the canister 115. For example, the controller 130 may determine that the canister is about 50% full and may communicate this value with the user interface 210 of the therapy unit 160.

FIG. 5C shows the canister 115 with a third volume of fluid or a third amount of fluid 508 in the sealed interior. The first sensor 264, the second sensor 266, the third sensor 268, the fourth sensor 270, the fifth sensor 272, the sixth sensor 274, the seventh sensor 276, the eighth sensor 278, and the ninth sensor 280 may each sense the fluid 502 in the sealed interior of the canister 115.

In operation, the probe of the first sensor 264, the probe of the second sensor 266, the probe of the third sensor 268, the probe of the fourth sensor 270, the probe of the fifth sensor 272, the probe of the sixth sensor 274, the probe of the seventh sensor 276, the probe of the eighth sensor 278, and the probe of the ninth sensor 280 may each sense the presence of the fluid 502 in a portion of their respective sensing paths. The first sensor 264, the second sensor 266, the third sensor 268, the fourth sensor 270, the fifth sensor 272, the sixth sensor 274, the seventh sensor 276, the eighth sensor 278, and the ninth sensor 280 may communicate a signal to the controller 130 indicative of the presence of fluid in their respective sensing paths. In some embodiments, the signal associated with each probe corresponding with a respective sensor may further indicate additional information collected about the third amount of fluid 508 in the sealed interior of the canister 115. The controller 130 may receive the signal from each sensor of the sensor array 145 and determine the third amount of fluid 508 in the sealed interior of the canister 115. For example, the controller 130 may determine that the canister is about 90% full and may communicate this value with the user interface 210 of the therapy unit 160.

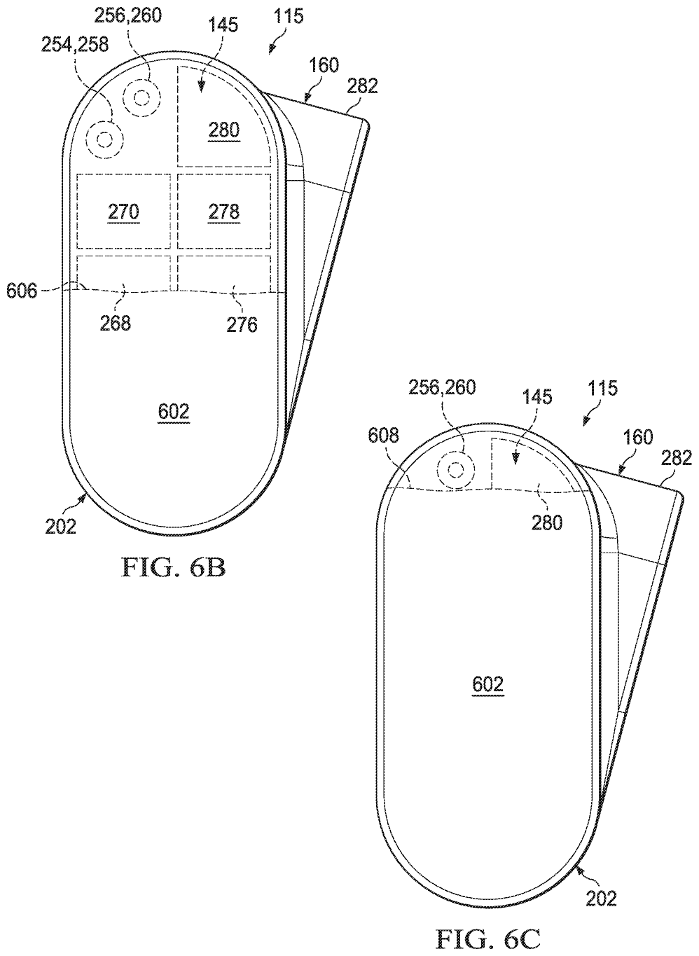

FIGS. 6A-6C are right-side views of example embodiments of the therapy unit 160 and the canister 115 with fluid 602 in the sealed interior of the canister 115. FIGS. 6A-6C show the therapy unit 160 and the canister 115 coupled together and in an orientation where the first edge 234 and the second edge 236 of the lid 204 of the canister 115 are perpendicular to a horizontal surface. In this orientation, the canister 115 is in a vertical orientation. In FIGS. 6A-6C, the controller 130 may be located within the therapy unit 160 and may be configured to determine an amount of the fluid 602 stored in the sealed interior of the canister 115.

FIG. 6A shows the canister 115 with a first volume of fluid or a first amount of fluid 604 in the sealed interior. The first sensor 264 and the fifth sensor 272 may each sense the fluid 602 in the sealed interior of the canister 115. The second sensor 266, the third sensor 268, the fourth sensor 270, the sixth sensor 274, the seventh sensor 276, the eighth sensor 278, and the ninth sensor 280 may not sense the fluid 602.

In operation, the probe of the second sensor 266, the probe of the third sensor 268, the probe of the fourth sensor 270, the probe of the sixth sensor 274, the probe of the seventh sensor 276, the probe of the eighth sensor 278, and the probe of the ninth sensor 280 may not sense the presence of the fluid 602 in each of their respective sensing paths. The second sensor 266, the third sensor 268, the fourth sensor 270, the sixth sensor 274, the seventh sensor 276, the eighth sensor 278, and the ninth sensor 280 may communicate a signal to the controller 130 indicative of the absence of fluid in each of the sensing paths of the second sensor 266, the third sensor 268, the fourth sensor 270, the sixth sensor 274, the seventh sensor 276, the eighth sensor 278, and the ninth sensor 280. The probe of the first sensor 264 and the probe of the fifth sensor 272 may each sense the presence of the fluid 602 in a portion of their respective sensing paths. The first sensor 264 and the fifth sensor 272 may then communicate a signal to the controller 130 indicative of the presence of fluid in their respective sensing paths. In some embodiments, the signal associated with each probe corresponding with a respective sensor may further indicate additional information collected about the first amount of fluid 604 in the sealed interior of the canister 115. The controller 130 may receive the signal from each sensor of the sensor array 145 and determine the first amount of fluid 604 in the sealed interior of the canister 115. For example, the controller 130 may determine that the canister is about 20% full and may communicate this value with the user interface 210 of the therapy unit 160.

FIG. 6B shows the canister 115 with a second volume of fluid or a second amount of fluid 606 in the sealed interior. The first sensor 264, the second sensor 266, the third sensor 268, the fifth sensor 272, the sixth sensor 274, and the seventh sensor 276 may each sense the fluid 602 in the sealed interior of the canister 115. The fourth sensor 270, the eighth sensor 278, and the ninth sensor 280 may not sense the fluid 602.

In operation, the probe of the fourth sensor 270, the probe of the eighth sensor 278, and the probe of the ninth sensor 280 may not sense the presence of the fluid 602 in each of their respective sensing paths. The fourth sensor 270, the eighth sensor 278, and the ninth sensor 280 may communicate a signal to the controller 130 indicative of the absence of fluid in each of the sensing paths of the fourth sensor 270, the eighth sensor 278, and the ninth sensor 280. The probe of the first sensor 264, the probe of the second sensor 266, the probe of the third sensor 268, the probe of the fifth sensor 272, the probe of the sixth sensor 274, and the probe of the seventh sensor 276 may each sense the presence of the fluid 602 in a portion of their respective sensing paths. The first sensor 264, the second sensor 266, the third sensor 268, fifth sensor 272, the sixth sensor 274, and the seventh sensor 276 may communicate a signal to the controller 130 indicative of the presence of fluid in their respective sensing paths. In some embodiments, the signal associated with each probe corresponding with a respective sensor may further indicate additional information collected about the second amount of fluid 606 in the sealed interior of the canister 115. The controller 130 may receive the signal from each sensor of the sensor array 145 and determine the second amount of fluid 606 in the sealed interior of the canister 115. For example, the controller 130 may determine that the canister is about 50% full and may communicate this value with the user interface 210 of the therapy unit 160.

FIG. 6C shows the canister 115 with a third volume of fluid or a third amount of fluid 608 in the sealed interior. The first sensor 264, the second sensor 266, the third sensor 268, the fourth sensor 270, the fifth sensor 272, the sixth sensor 274, the seventh sensor 276, the eighth sensor 278, and the ninth sensor 280 may each sense the fluid 602 in the sealed interior of the canister 115.

In operation, the probe of the first sensor 264, the probe of the second sensor 266, the probe of the third sensor 268, the probe of the fourth sensor 270, the probe of the fifth sensor 272, the probe of the sixth sensor 274, the probe of the seventh sensor 276, the probe of the eighth sensor 278, and the probe of the ninth sensor 280 may each sense the presence of the fluid 602 in a portion of their respective sensing paths. The first sensor 264, the second sensor 266, the third sensor 268, the fourth sensor 270, the fifth sensor 272, the sixth sensor 274, the seventh sensor 276, the eighth sensor 278, and the ninth sensor 280 may communicate a signal to the controller 130 indicative of the presence of fluid in their respective sensing paths. In some embodiments, the signal associated with each probe corresponding with a respective sensor may further indicate additional information collected about the third amount of fluid 608 in the canister 115. The controller 130 may receive the signal from each sensor of the sensor array 145 and determine the third amount of fluid 608 in the sealed interior of the canister 115. For example, the controller 130 may determine that the canister is about 90% full and may communicate this value with the user interface 210 of the therapy unit 160.

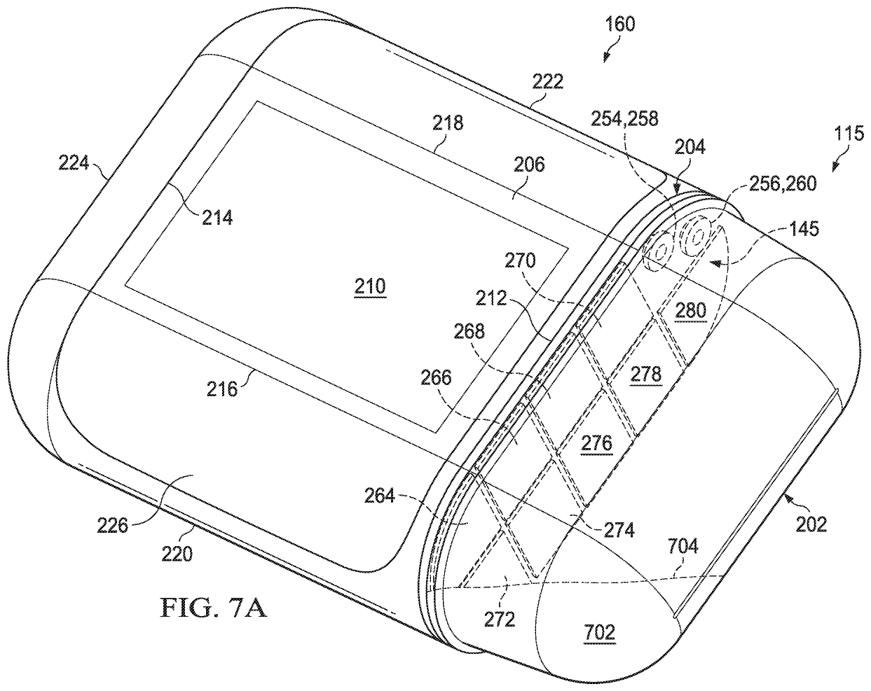

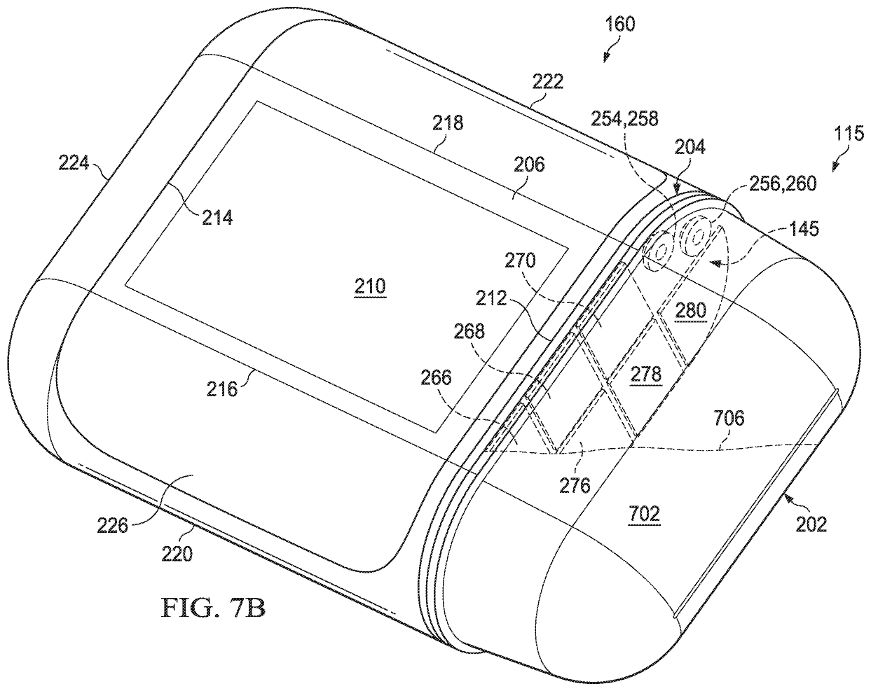

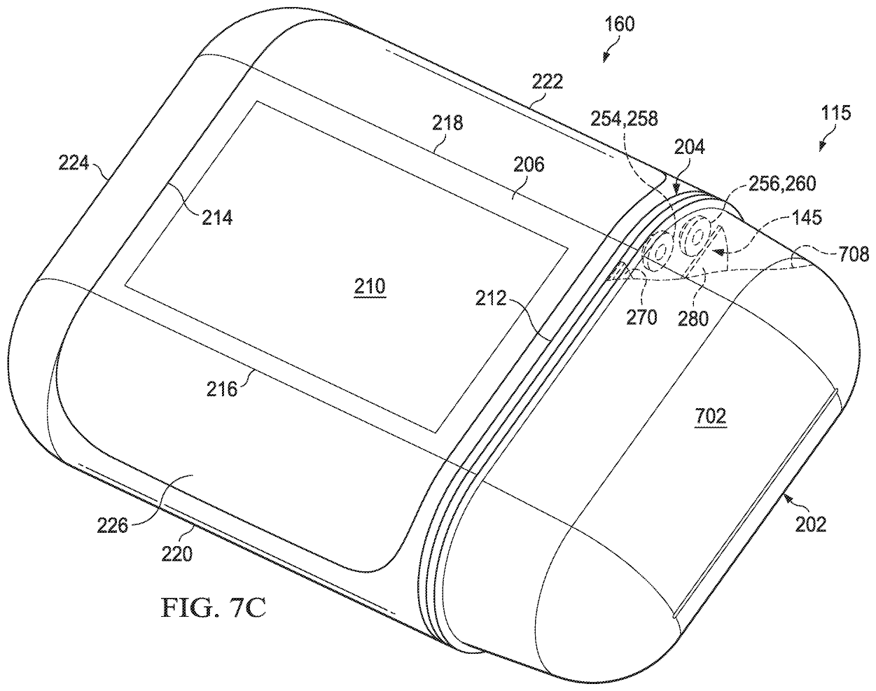

FIGS. 7A-7C are perspective views of example embodiments of the therapy unit 160 and the canister 115 with fluid 702 in the sealed interior of the canister 115. FIGS. 7A-7C show the therapy unit 160 and the canister 115 coupled together and in a complex orientation. For example, the therapy unit 160 and the canister 115 may be held in an orientation that is convenient for the user. In other embodiments, the therapy unit 160 and the canister 115 may be in transportation from a first location to a second location. In FIGS. 7A-7C, the controller 130 may be located within the therapy unit 160 and may be configured to determine an amount of the fluid 602 stored in the sealed interior of the canister 115. In this orientation, the fluid 702 in the sealed interior of the canister 115 may be moving around in the sealed interior and the amount of fluid in the sealed interior may not be able to be accurately determined. When an accurate amount of fluid cannot be determined, the controller 130 may determine a fill level range for the amount of fluid in the sealed interior of the canister 115.

FIG. 7A shows the canister 115 with a first volume of fluid or a first amount of fluid 704 in the scaled interior. The first sensor 264, second sensor 266, the third sensor 268, the fifth sensor 272, the sixth sensor 274, and the seventh sensor 276 may each sense the fluid 702 in the sealed interior of the canister 115. The fourth sensor 270, the eighth sensor 278, and the ninth sensor 280 may not sense the fluid 702.

In operation, the probe of the fourth sensor 270, the probe of the eighth sensor 278, and the probe of the ninth sensor 280 may not sense the fluid 702 in each of their respective sensing paths. The fourth sensor 270, the eighth sensor 278, and the ninth sensor 280 may communicate a signal to the controller 130 indicative of the absence of fluid in each of the sensing paths of the fourth sensor 270, the eighth sensor 278, and the ninth sensor 280. The probe of the first sensor 264, the probe of the second sensor 266, the probe of the third sensor 268, the probe of the fifth sensor 272, the probe of the sixth sensor 274, and the probe of the seventh sensor 276 may each sense the presence of the fluid 602 in a portion of their respective sensing paths. The first sensor 264, the second sensor 266, the third sensor 268, the fifth sensor 272, the sixth sensor 274, and the seventh sensor 276 may communicate a signal to the controller 130 indicative of the presence of fluid in their respective sensing paths. In some embodiments, the signal associated with each probe corresponding with a respective sensor may further indicate additional information collected about the first amount of fluid 704 in the scaled interior of the canister 115. The controller 130 may receive the signal from each sensor of the sensor array 145 and determine the first amount of fluid 704 in the sealed interior of the canister 115. For example, the controller 130 may determine that the canister is about 20% full and may communicate this value with the user interface 210 of the therapy unit 160.

FIG. 7B shows the canister 115 with a second volume of fluid or a second amount of fluid 706 in the sealed interior. The first sensor 264, the second sensor 266, the third sensor 268, the fourth sensor 270, the fifth sensor 272, the sixth sensor 274, the seventh sensor 276, and the eighth sensor 278 may each sense the fluid 602 in the sealed interior of the canister 115. The ninth sensor 280 may not sense the fluid 702.

In operation, the probe of the ninth sensor 280 may not sense the presence of the fluid 602 in its sensing path. The ninth sensor 280 may communicate a signal to the controller 130 indicative of the absence of fluid in the sensing path of the ninth sensor 280. The probe of the first sensor 264, the probe of the second sensor 266, the probe of the third sensor 268, the probe of the fourth sensor 270, the probe of the fifth sensor 272, the probe of the sixth sensor 274, the probe of the seventh sensor 276, and the probe of the eighth sensor 278 may each sense the presence of the fluid 702 in a portion of their respective sensing paths. The first sensor 264, the second sensor 266, the third sensor 268, the fourth sensor 270, the fifth sensor 272, the sixth sensor 274, the seventh sensor 276, and the eighth sensor 278 may communicate a signal to the controller 130 indicative of the presence of fluid in their respective sensing paths. In some embodiments, the signal associated with each probe corresponding with a respective sensor may further indicate additional information collected about the second amount of fluid 706 in the sealed interior of the canister 115. The controller 130 may receive the signal from each sensor of the sensor array 145 and determine the second amount of fluid 706 in the sealed interior of the canister 115. For example, the controller 130 may determine that the canister is about 50% full and may communicate this value with the user interface 210 of the therapy unit 160.

FIG. 7C shows the canister 115 with a third volume of fluid or a third amount of fluid 708 in the sealed interior. The first sensor 264, the second sensor 266, the third sensor 268, the fourth sensor 270, the fifth sensor 272, the sixth sensor 274, the seventh sensor 276, the eighth sensor 278, and the ninth sensor 280 may each sense the fluid 702 in the sealed interior of the canister 115.

In operation, the probe of the first sensor 264, the probe of the second sensor 266, the probe of the third sensor 268, the probe of the fourth sensor 270, the probe of the fifth sensor 272, the probe of the sixth sensor 274, the probe of the seventh sensor 276, the probe of the eighth sensor 278, and the probe of the ninth sensor 280 may each sense the presence of the fluid 602 in a portion of their respective sensing paths. The first sensor 264, the second sensor 266, the third sensor 268, the fourth sensor 270, the fifth sensor 272, the sixth sensor 274, the seventh sensor 276, the eighth sensor 278, and the ninth sensor 280 may then communicate a signal to the controller 130 indicative of the presence of fluid in their respective sensing paths. In some embodiments, the signal associated with each probe corresponding with a respective sensor may further indicate additional information collected about the third amount of fluid 708 in the sealed interior of the canister 115. The controller 130 may receive the signal from the each sensor of the sensor array 145 and determine the third amount of fluid 708 in the sealed interior of the canister 115. For example, the controller 130 may determine that the canister is about 90% full and may communicate this value with the user interface 210 of the therapy unit 160.

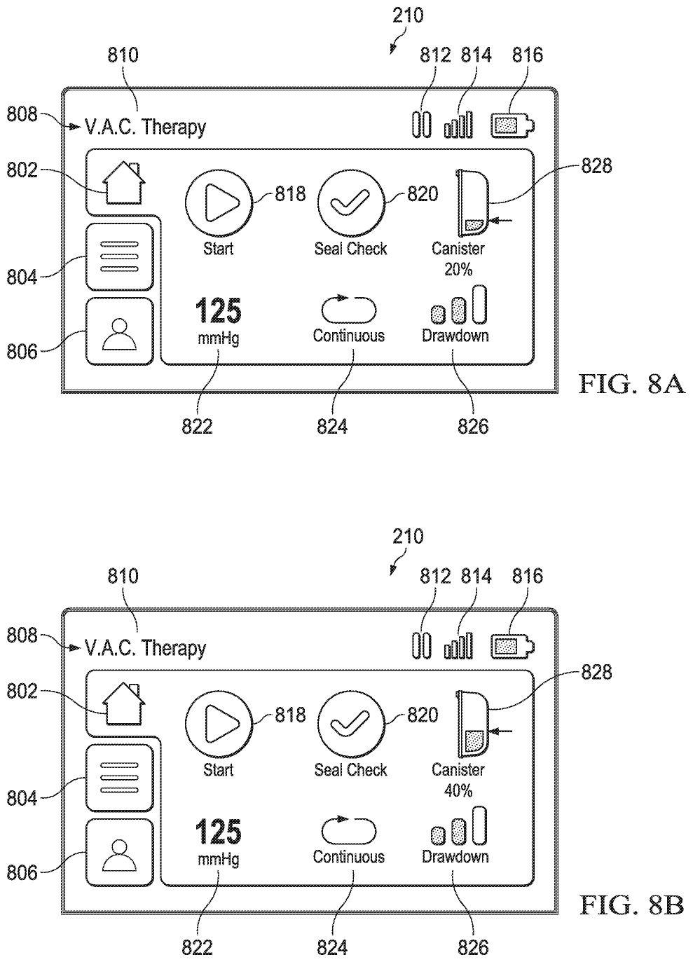

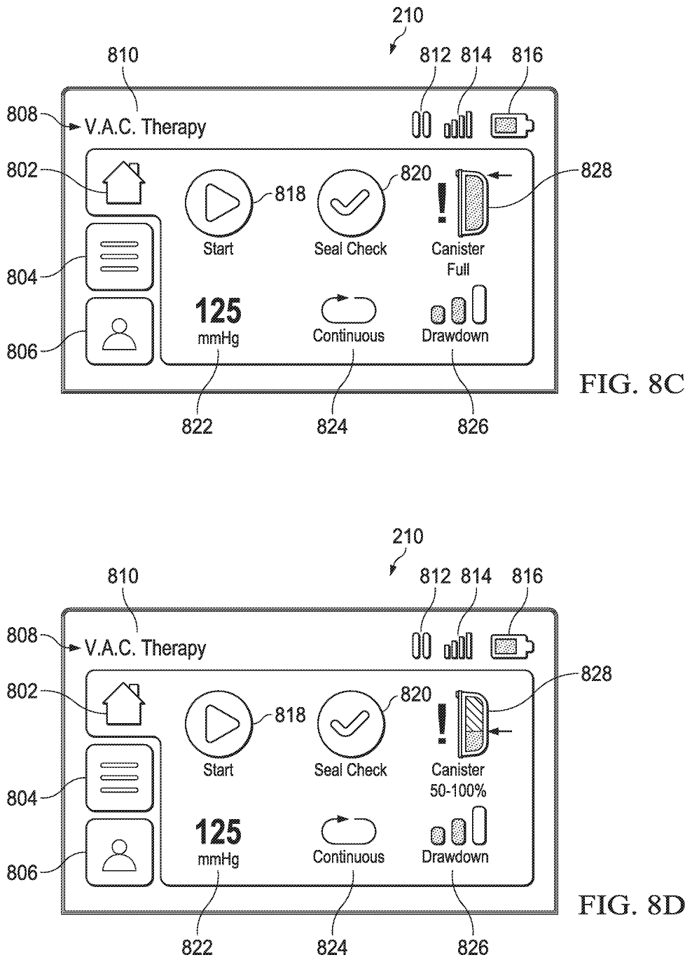

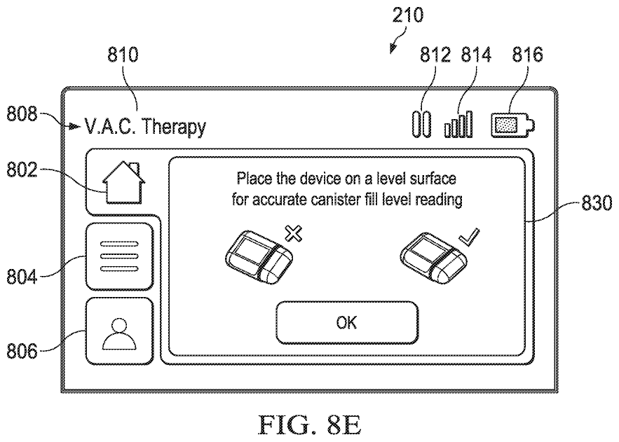

FIG. 8A is an example embodiment of the user interface 210 that may be associated with some embodiments of the therapy unit 160. In some embodiments, the user interface 210 may be a touch screen. In other embodiments, the user interface 210 may be a screen having buttons permitting a user to navigate through different menus and options of the user interface 210. The user interface 210 may be configured to output messages related to the canister 115. For example, the user interface 210 may output a message associated with the value of the parameter of the canister 115. In some embodiments, the parameter of the canister may be the fill level of the canister 115. The user interface 210 may have a home screen 802, a settings menu 804, and a user information screen 806. In other embodiments, there may be additional or different screens included on the user interface 210. The user interface may also comprise a top ribbon or banner 808. In some embodiments, the banner may display a title 810. For example, the title 810 may be “V.A.C. Therapy” indicating that the therapy system is delivering Vacuum Assisted Closure Therapy to a user. The banner may also include a status icon 812. The status icon 812 may show whether negative-pressure therapy is being applied to the dressing 110 or whether negative-pressure therapy is paused. In some embodiments, the therapy system 100 may operate wirelessly and may be connected to a wireless network. In these embodiments, there may be a signal strength icon 814 included in the banner 808. The banner 808 may also display a battery icon 816. The battery icon 816 may indicate a battery percentage remaining associated with a power source included in the therapy unit 160.

The home screen 802 may include a start icon 818 configured to initiate a therapy process, such as negative-pressure therapy. In some embodiments, there may be a pause icon that replaces the start icon 818 if negative-pressure therapy is active to allow a medical professional or a user to pause the negative-pressure therapy.

The home screen 802 of the user interface 210 may also include a seal check icon 820. The seal check icon 820 may be configured to indicate if the therapy system 100 is scaled properly at various locations. For example, the seal check icon 820 may indicate if the therapy unit 160 is sealed to the canister 115 and if the canister 115 is sealed to the dressing 110. If there is not a sufficient seal between the therapy unit 160 and the canister 115 or between the canister 115 and the dressing 110, the seal check icon 820 may appear in a different color, it may notify the user with a sound, or it may include a warning icon or any other method of indicating that that there is an insufficient seal. In some embodiments, the seal check icon 820 may be a button that may be pressed to start and stop flow rate monitoring which may give a measured flow reading. The user interface 210 may display a bar graph or may output an audible signal that may change in pitch corresponding to the measured flow reading. For example, if there is a leak detected in the therapy system 100, the measured flow reading may be high and the measured flow rate may increase corresponding to the size of the leak in the therapy system 100. The seal check icon 820 may therefore be used to determine if the therapy system 100 has been set up correctly to deliver negative pressure therapy to a patient.

The home screen 802 of the user interface 210 may further include a pressure display icon 822. The pressure display icon 822 may indicate a therapy pressure of the dressing. In other embodiments, the pressure display icon 822 may indicate a current pressure at the dressing 110. The home screen 802 of the user interface 210 may also include a therapy style icon 824. The therapy style icon 824 may indicate whether continuous negative-pressure, intermittent negative-pressure, or a different kind of therapy is selected. The home screen 802 of the user interface 210 may also include a drawdown status icon 826. The drawdown status icon 826 may indicate how close the therapy unit 160 is to generating the therapy pressure.

The home screen 802 of the user interface 210 may further include a fill level icon 828. The fill level icon 828 may be configured to display a fill level or a fill level range of the canister 115. In some embodiments, the fill level may be displayed by default. In other embodiments, the controller 130 may be unable to determine the fill level. If the controller 130 is unable to determine the fill level, the controller 130 may determine the fill level range and display the fill level range on the user interface 210. The fill level range may be an estimate of the amount of fluid stored in the canister 115. The fill level range may be determined by prior fill level values. In some embodiments, historical data associated with the canister 115 or other therapy systems 100 may also be included in the fill level range calculations done by the controller 130. The fill level icon 828 may appear as a bar that may be filled in by an amount corresponding to the fill level of the canister 115. For example, if the canister 115 is 20% full, the fill level icon 828 may appear 20% filled in. In some embodiments, an arrow may be included beside the bar of the fill level icon 828 pointing to the top of the shaded area of the bar. The fill level icon 828 may also display in words the fill level of the canister 115. For example, the fill level icon 828 may include the words “Canister 20%” to indicate that the canister 115 is 20% full.

FIG. 8B is another example of the user interface 210 that can be associated with some embodiments of the therapy system 100. The user interface 210 may appear substantially as described above for FIG. 8A. The fill level icon 828 of FIG. 8B is indicating that the canister 115 is 40% full. The fill level icon 828 appears 40% full and the words under the bar of the fill level icon 828 say “Canister 40%.”

FIG. 8C is another example of the user interface 210 that can be associated with some embodiments of the therapy system 100. The user interface 210 may appear substantially as described above for FIGS. 8A and 8B. In this embodiment, the fill level icon 828 is indicating that the canister 115 is full. The bar of the fill level icon 828 is completely filled in and the words under the bar say “Canister Full.” When the fill level icon 828 indicates that the canister 115 is full, there may be an additional symbol that appears in the fill level icon 828 such as an exclamation point. In other embodiments, when the canister 115 is full, there may be a sound, a vibration, or another way that the user or the medical professional is notified that the canister 115 is full.

FIG. 8D is another example of the user interface 210 that can be associated with some embodiments of the therapy system 100. The user interface 210 may appear substantially as described above for FIGS. 8A, 8B, and 8C. In this embodiment, the fill level icon 828 is displaying a fill level range and is indicating that the canister 115 is 50%-100% full. Because the fill level icon 828 is displaying a fill level range, the controller 130 may have been unable to determine an accurate fill level of the canister 115. The controller 130 may have determined the fill level range by using prior fill level values of the canister 115. For example, if the last fill level of the canister 115 was 40%, the controller 130 may have determined that an additional amount of fluid has entered the canister 115 and the fill level range is greater than 40%. Because the fill level icon 828 is indicating that the canister 115 could be 100% full, the exclamation point may be included in the fill level icon 828 to fully inform the user of the potential that the canister 115 is completely full.

FIG. 8E is an example of an information screen 830 of the user interface 210. In some embodiments, the information screen may appear automatically when the controller 130 cannot determine the fill level of the canister 115. In other embodiments, the information screen 830 may appear after the user selects the fill level icon 828 by touching or pressing the fill level icon 828 when the fill level icon 828 is displaying a fill level range of the canister 115. The information screen 830 may display a message indicating that the canister 115 should be placed on a level surface so the controller 130 can determine an accurate fill level of the canister 115. The information screen 830 may display images of the therapy unit 160 and the canister 115 in an optimal orientation with a check mark and in a non-optimal orientation with an “X”. The information screen 830 may also have an “OK” button that can be selected to move from the information screen 830 back to the home screen 802.

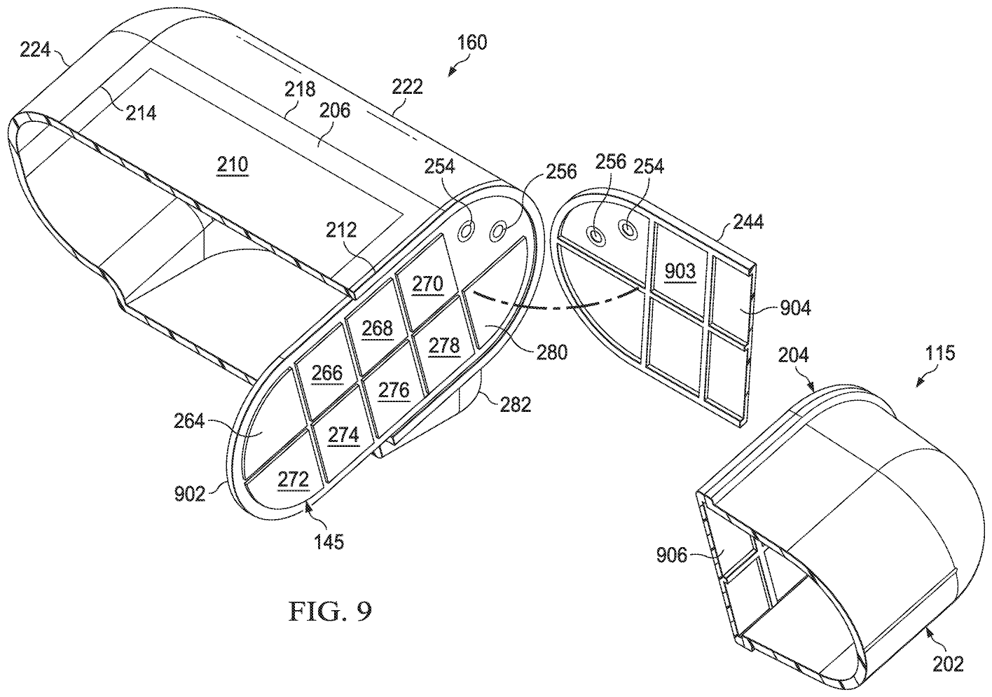

FIG. 9 is a cut-away view of an example embodiment of the therapy unit 160, the sensor array 145, and the canister 115 that can be associated with some embodiments of the therapy system 100. In some embodiments, the sensor array 145 may be coupled to a printed circuit board 902. The sensor array 145 may be coupled to the printed circuit board 902 to allow easy communication between the sensor array 145 and the controller 130. In other embodiments, the sensor array 145 may not be coupled to the printed circuit board 902 and may be coupled to the third wall 244 of the therapy unit 160 or may be integrated into the third wall 244 of the therapy unit 160. The printed circuit board 902 having the sensor array 145 may be positioned proximate to the interior surface 903 of the third wall 244 of the therapy unit 160 such that each probe of each sensor of the sensor array 145 is facing the exterior surface 242 of the third wall 244 of the therapy unit 160.

In some example embodiments, a Cypress system may be used for the sensor array 145 and the printed circuit board 902. For example, the sensor array 145 may be a CY8CKIT-022 CapSense® Liquid Level Sensing Shield and the printed circuit board 902 may be an associated flex sensor printed circuit board. The flex sensor printed circuit board may be a capacitive strip that may have a variable capacitance across the capacitive strip. For example, the capacitance may vary in magnitude as fluid and exudate flow into the canister 115. In embodiments where the Cypress system is used, the canister 115 may be a non-conductive canister where the fluid in the canister is a dielectric for the system. In other embodiments, the sensor array 145 may be formed from the sensors described above with reference to FIG. 3.

In some embodiments, the third wall 244 of the therapy unit 160 may include at least one cavity or region of localized thinning 904. There may be a region of localized thinning 904 that corresponds to each of the first sensor 264, the second sensor 266, the third sensor 268, the fourth sensor 270, the fifth sensor 272, the sixth sensor 274, the seventh sensor 276, the eighth sensor 278, and the ninth sensor 280. Each region of localized thinning 904 may allow each sensor of the sensor array 145 to more accurately sense the fluid in the canister 115.

The lid 204 of the canister 115 may also comprise at least one cavity or region of localized thinning 906. There may be a region of localized thinning 906 that corresponds to each of the first sensor 264, the second sensor 266, the third sensor 268, the fourth sensor 270, the fifth sensor 272, the sixth sensor 274, the seventh sensor 276, the eighth sensor 278, and the ninth sensor 280. Each region of localized thinning 906 of the lid 204 may be aligned with the corresponding region of localized thinning 904 of the third wall 244 of the therapy unit 160. Each region of localized thinning 906 of the lid 204 may reduce the amount of material through which each sensor of the sensor array 145 senses the fluid in the canister 115. For example, without each region of localized thinning 906 and each region of localized thinning 904, each sensor of the sensor array may have to function through the entire thickness of the third wall 244 and the lid 204 which may be about 6 millimeters. With each region of localized thinning 906 and each region of localized thinning 904, each sensor of the sensor array 145 may function through about 3 millimeters between the third wall 244 and the lid 204. Each region of localized thinning 906 of the lid 204 may be uniform in thickness over the area corresponding to each sensor of the sensor array 145 to provide a consistent orientation for each sensor of the sensor array 145. Similarly, each region of localized thinning 904 of the third wall 244 of the therapy unit 160 may be uniform in thickness over the area corresponding to each sensor of the sensor array 145 to provide a consistent orientation for each sensor of the sensor array 145. Having a uniform thickness for each region of localized thinning 904 of the third wall 244 of the therapy unit 160 may also provide a high quality surface for coupling the sensor array 145 to the third wall 244 of the therapy unit 160. In some embodiments, each region of localized thinning 906 of the lid 204 and each region of localized thinning 904 of the third wall 244 of the therapy unit 160 may have a thickness corresponding to an expected maximum operating distance of each sensor of the sensor array 145 to create an optimal environment for the sensor array 145 to detect an amount of fluid in the canister 115. In some embodiments, each region of localized thinning 904 of the third wall 244 of the therapy unit 160 may be thinner than each region of localized thinning 906 of the lid 204 to maintain the structural integrity of the canister 115 during the application of negative pressure therapy.

In some embodiments, the third wall 244 of the therapy unit 160 may include the regions of localized thinning 904 and the lid 204 may not include the regions of localized thinning 906. In other embodiments, the third wall 244 of the therapy unit 160 may not include the regions of localized thinning 904 and the lid 204 may include the regions of localized thinning 906. In still other embodiments, the third wall 244 of the therapy unit 160 may include the regions of localized thinning 904 and the lid 204 may include the regions of localized thinning 906 or the third wall 244 of the therapy unit 160 may not include the regions of localized thinning 904 and the lid 204 may not include the regions of localized thinning 906.

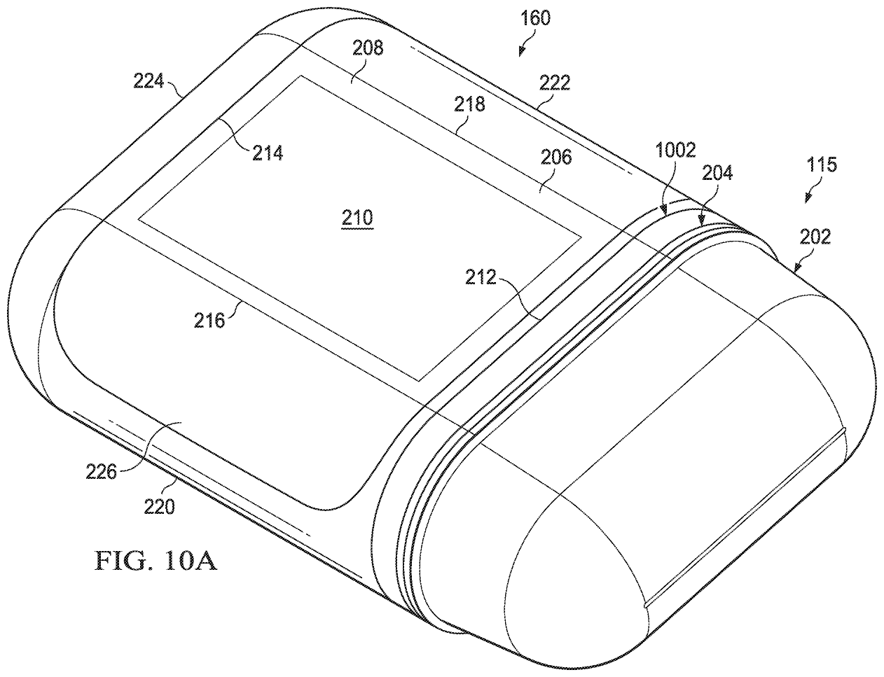

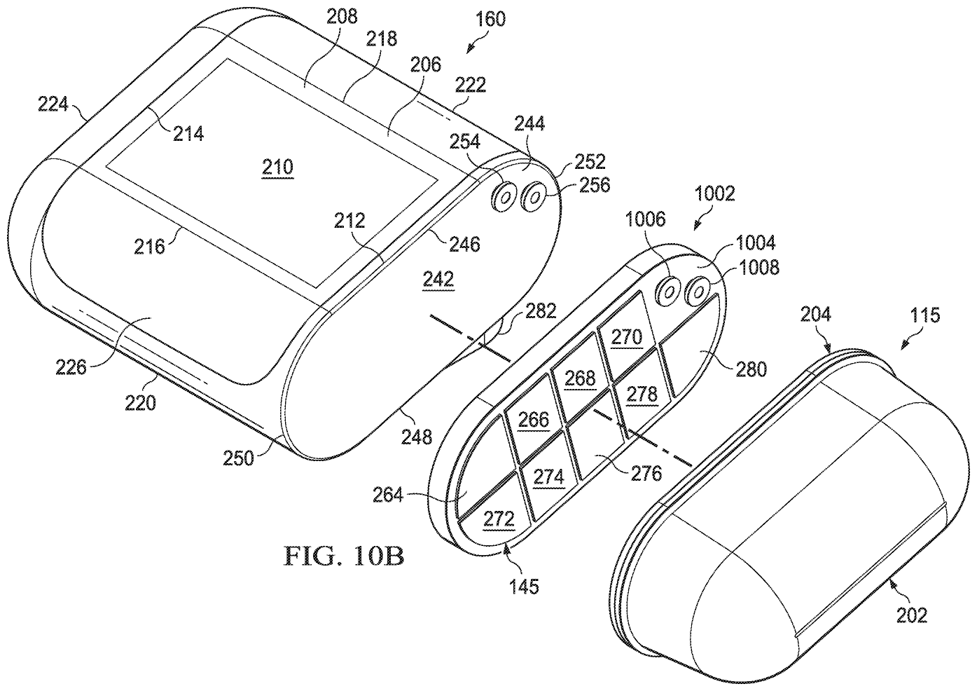

FIG. 10A is a perspective view of another example embodiment of the therapy unit 160, the sensor array 145, and the canister 115. In some embodiments, the sensor array 145 may be a component of a sensor module 1002. The therapy unit and the canister may be substantially as described above with reference to FIGS. 2 and 3 and the sensor array 145 may substantially as described above with reference to FIG. 3. The sensor module 1002 may be disposed between the third wall 244 of the therapy unit 160 and the lid 204 of the canister 115. The sensor module 1002 may be communicatively coupled to the controller 130 to provide communication between each sensor of the sensor array 145 and the controller 130.

FIG. 10B is an exploded view of the therapy unit 160, the sensor module 1002, and the canister 115 of FIG. 10A. The sensor module 1002 may have a first wall 1004 that may be substantially the same size and shape as the third wall 244 of the therapy unit 160 and the lid 204 of the canister 115. The sensor module 1002 may have a second wall opposite the first wall 1004. The first wall 1004 of the sensor module 1002 may be configured to couple to the lid 204 of the canister 115 and the second wall of the sensor module 1002 may be configured to couple to the third wall 244 of the therapy unit 160.

The sensor array 145 may include the first sensor 264, the second sensor 266, the third sensor 268, the fourth sensor 270, the fifth sensor 272, the sixth sensor 274, the seventh sensor 276, the eighth sensor 278, and the ninth sensor 280 substantially as described above. The sensor array 145 may be coupled to the first wall 1004 of the sensor module 1002 or may be formed integrally with the first wall 1004 of the sensor module 1002. Each probe of each sensor of the sensor array 145 may be directed towards the sealed interior of the canister 115 as described above.

The sensor module 1002 may also include a fifth connection point 1006 and a sixth connection point 1008. The fifth connection point 1006 may be configured to align with the first connection point 254 and the third connection point 258. The sixth connection point 1008 may be configured to align with the second connection point 256 and the fourth connection point 260. The fifth connection point 1006 and the sixth connection point 1008 may work with the first connection point 254, the second connection point 256, the third connection point 258, and the fourth connection point 260 to provide communication between the therapy unit 160 and the canister 115. The sensor module 1002 may be comprised of any of the materials listed above for the canister 115.

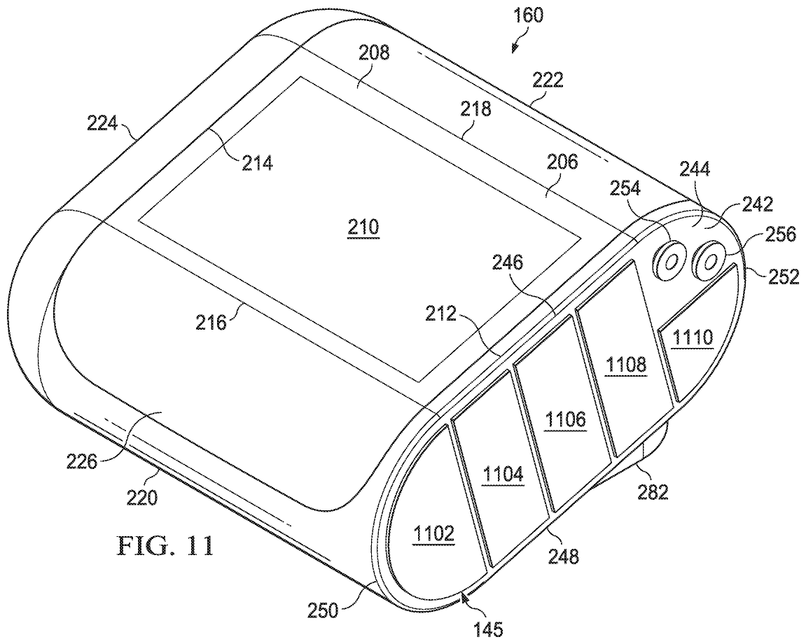

FIG. 11 is a perspective view of the therapy unit 160 of any of the above figures with an alternate embodiment of the sensor array 145. The sensor array 145 may be coupled to the third wall 244 of the therapy unit 160 as described above. In some example embodiments, the sensor array 145 may include a first sensor 1102, a second sensor 1104, a third sensor 1106, a fourth sensor 1108, and a fifth sensor 1110. The first sensor 1102, the second sensor 1104, the third sensor 1106, the fourth sensor 1108, and the fifth sensor 1110 may extend from the first edge 246 of the third wall 244 to the second edge 248 of the third wall 244 of the therapy unit 160. The first sensor 1102 may be in the shape of a semi-circle and may be proximate to the third edge 250 of the third wall 244 of the therapy unit 160. The second sensor 1104, the third sensor 1106, and the fourth sensor 1108 may be rectangular. The second sensor 1104 may be between the first sensor 1102 and the third sensor 1106, the third sensor 1106 may be between the second sensor 1104 and the fourth sensor 1108, and the fourth sensor 1108 may be between the third sensor 1106 and the fifth sensor 1110. The fifth sensor 1110 may be in the shape of a sector with a radius approximately equal to the radius of the semicircle defined by the fourth edge 252 of the third wall 244 of the therapy unit 160. The fifth sensor 1110 may be proximate to the fourth sensor 1108, the fourth edge 252 of the therapy unit 160, and the second edge 248 of the therapy unit 160. Each sensor of the sensor array 145 may function substantially as described above with reference to FIGS. 3, 5A-C, 6A-C, and 7A-C.

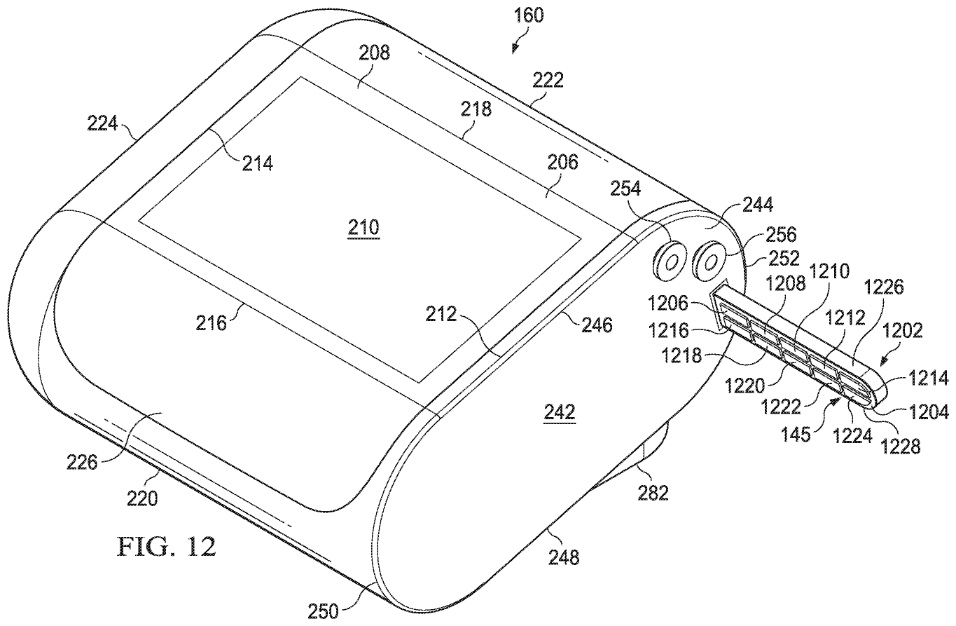

FIG. 12 is a perspective view of the therapy unit 160 of FIG. 1. In some embodiments, the therapy unit 160 may include an extension 1202 protruding from the exterior surface 242 of the third wall 244 of the therapy unit 160. The extension 1202 may have a first surface 1204 and a second surface opposite the first surface 1204. The sensor array 145 may be coupled to the first surface 1204 of the extension 1202 or it may be formed integrally with the first surface 1204 of the extension 1202. The first surface 1204 may couple to an exterior of the canister body 202 when the canister 115 is coupled to the therapy unit 160. When the first surface 1204 of the extension 1202 is coupled to the exterior of the canister body 202, each sensor of the sensor array 145 may be directed towards the sealed interior of the canister 115.

The sensor array 145 may include a first sensor 1206, a second sensor 1208, a third sensor 1210, a fourth sensor 1212, a fifth sensor 1214, a sixth sensor 1216, a seventh sensor 1218, an eighth sensor 1220, a ninth sensor 1222, and a tenth sensor 1224. Each sensor may be rectangular in shape and may be communicatively coupled to the controller 130 of the therapy unit 160. The first sensor 1206 may be proximate to the third wall 244 of the therapy unit 160, the second sensor 1208, and a first edge 1226 of the first surface 1204 of the extension 1202. The second sensor 1208 may be proximate to the first edge 1226 and may be between the first sensor 1206 and the third sensor 1210. The third sensor 1210 may be proximate to the first edge 1226 and may be between the second sensor 1208 and the fourth sensor 1212. The fourth sensor 1212 may be proximate to the first edge 1226 and may be between the third sensor 1210 and the fifth sensor 1214. The fifth sensor 1214 may be proximate to the first edge 1226 and the fourth sensor 1212. The sixth sensor 1216 may be proximate to the third wall 244 of the therapy unit 160 and a second edge 1228 of the first surface 1204 of the extension 1202. The sixth sensor 1216 may be below the first sensor 1206 and may be proximate to the seventh sensor 1218. The seventh sensor 1218 may be below the second sensor 1208, between the sixth sensor 1216 and the eighth sensor 1220, and proximate to the second edge 1228. The eighth sensor 1220 may be below the third sensor 1210, between the seventh sensor 1218 and the ninth sensor 1222, and proximate to the second edge 1228. The ninth sensor 1222 may be below the fourth sensor 1212, between the eighth sensor 1220 and the tenth sensor 1224, and proximate to the second edge 1228. The tenth sensor 1224 may be below the fifth sensor 1214 and proximate to the ninth sensor 1222 and the second edge 1228. Each sensor of the sensor array 145 may have a probe that may be directed towards the scaled interior of the canister 115. Each sensor of the sensor array 145 may function substantially the same as described above with reference to FIGS. 3, 5A-C, 6A-C, and 7A-C.

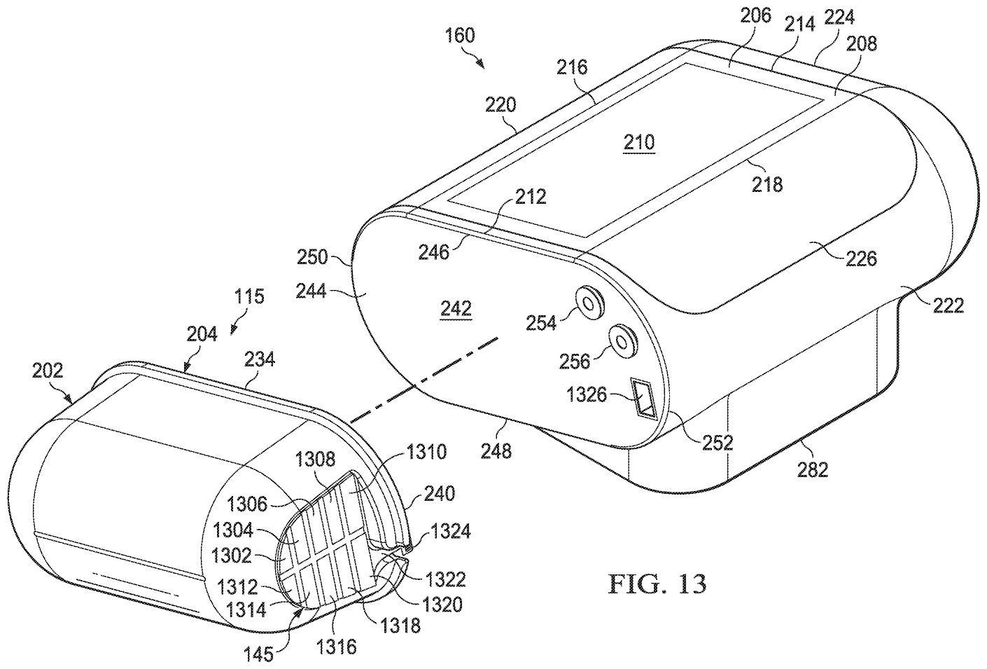

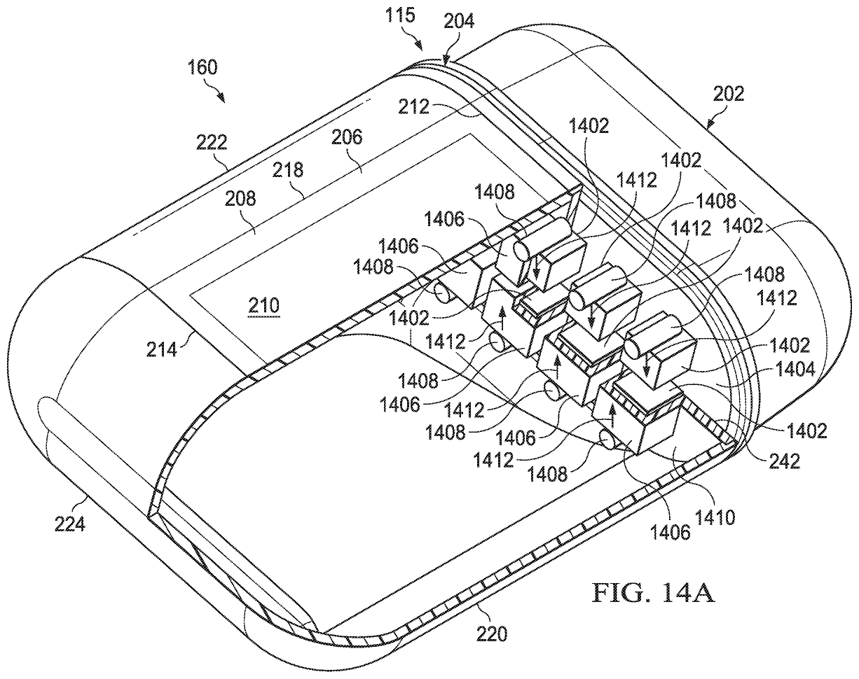

FIG. 13 is an exploded view of an alternate embodiment of the therapy unit 160 and the canister 115 of FIG. 1. In some embodiments, the sensor array 145 may be integrated into the canister 115. The sensor array 145 may include a first sensor 1302, a second sensor 1304, a third sensor 1306, a fourth sensor 1308, a fifth sensor 1310, a sixth sensor 1312, a seventh sensor 1314, an eighth sensor 1316, a ninth sensor 1318, and a tenth sensor 1320. In some embodiments, each sensor may be coupled to a printed circuit board 1322. The printed circuit board 1322 and the sensor array 145 may be coupled to the canister body 202 of the canister 115 or maybe formed integrally with the canister body 202. In some embodiments, the sensor array 145 may be located on the canister body 202 proximate to the fourth edge 240 of the lid 204 of the canister 115. In other embodiments, the printed circuit board 1322 and the sensor array 145 may be coupled to the lid 204 of the canister 115 or may be formed integrally with the lid 204 of the canister 115. Each sensor of the sensor array 145 may have a probe that may be directed towards the scaled interior of the canister 115. Each sensor of the sensor array 145 may function substantially the same as described above with reference to FIGS. 3, 5A-C, 6A-C, and 7A-C.

The canister 115 may have a projection 1324 that is coupled to the sensor array 145. The projection 1324 may be configured to connect to the therapy unit 160 through an opening 1326. The opening 1326 may be configured to receive the projection 1324 to allow the sensor array 145 to be coupled to the controller 130. The projection 1324 may extend from the canister 115 away from the canister body 202 and the lid 204. The opening 1326 may be located through the third wall 244 of the therapy unit 160 proximate to the fourth edge 252 of the third wall 244. In other embodiments, the projection 1324 and the opening 1326 may be at different locations but may still allow communication between the sensor array 145 and the controller 130 of the therapy unit 160.

In some embodiments, the canister 115 may have additional capabilities when the sensor array 145 is integrated into the canister 115. For example, the sensor array 145 may be able to sense additional information about the canister 115 such as identification of the canister 115, whether the canister has been tampered with, whether the canister 115 has been or can be reused, and what kind of therapy is being delivered to the dressing 110 by the therapy unit 160. This additional functionality may allow the canister 115 to communicate more information with the controller 130 and ultimately the user through the user interface 210.

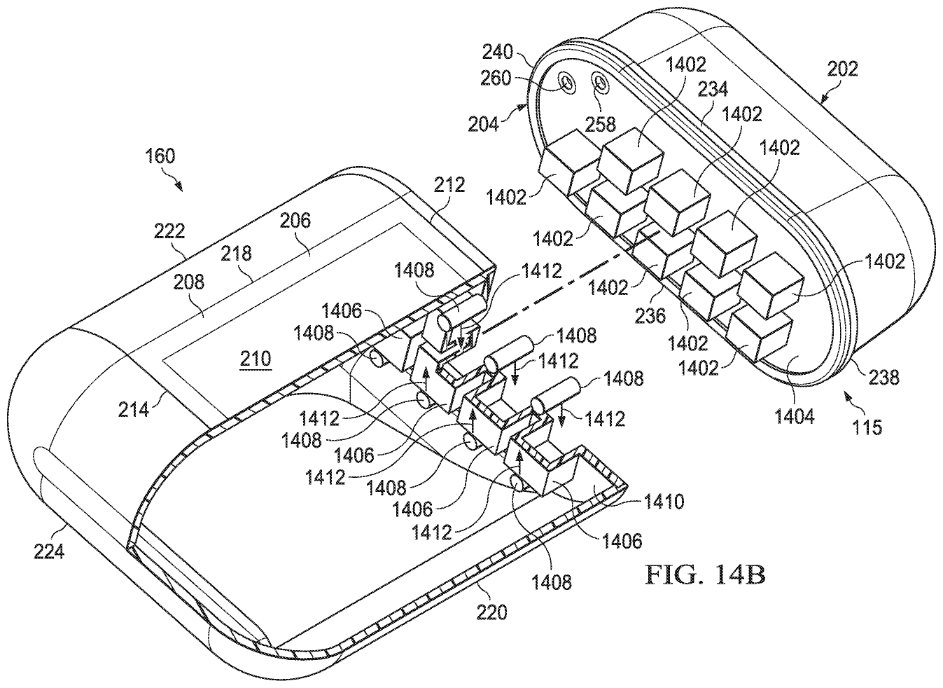

FIGS. 14A and 14B depict another embodiment of the therapy unit 160 and the canister 115 of FIG. 1. FIG. 14A is a perspective, partial cut-away view of the therapy unit 160 coupled to the canister 115. FIG. 14B is an exploded view of the partial cut-away view of the therapy unit 160 and the canister 115. The lid 204 of the canister 115 may comprise a plurality of protrusions 1402 extending from the exterior surface 1404 of the lid 204 of the canister 115. The plurality of protrusions 1402 may extend away from the first surface 230 of the lid 204 of the canister 115. Each protrusion 1402 of the plurality of protrusions 1402 may be received by a recess 1406 of the third wall 244 of the therapy unit 160. In some embodiments, each protrusion 1402 of the plurality of protrusions 1402 may be rectangular. In other embodiments, each protrusion 1402 of the plurality of protrusions 1402 may be circular, ovular, or a different shape and each recess 1406 may be a similar shape to receive the corresponding protrusion 1402.

In some embodiments, there may be a plurality of sensors 1408 positioned proximate to the interior surface 1410 of the third wall 244 of the therapy unit 160. Each sensor 1408 of the plurality of sensors 1408 may have a probe that may be directed towards one of the recesses 1406 of the third wall 244 of the therapy unit 160. Each probe of each sensor 1408 may be configured to sense the parameter associated with the canister 115 along a sensing path 1412 through the corresponding recess 1406 of the third wall 244 and the protrusion 1402 of the lid 204. In some embodiments, the parameter associated with the canister 115 may be the amount of fluid in the canister 115. In other embodiments, the parameter associated with the canister 115 may be the color of the fluid in the canister 115. In still other embodiments, the plurality of sensors 1408 may be configured to sense both the amount of fluid in the canister 115 and the color of the fluid in the canister 115.

In some embodiments, the plurality of sensors 1408 may be configured to sense the presence of fluid within the corresponding protrusion 1402 of the lid 204 of the canister 115. In some embodiments, each sensor 1408 of the plurality of sensors 1408 may be a non-contact capacitive sensor as described above. In other embodiments, each sensor 1408 of the plurality of sensors 1408 may be an air bubble fluid sensor. Each air bubble fluid sensor may be configured to determine the lack of a bubble or air located in the corresponding protrusion 1402 of the lid 204. If the air bubble fluid sensor determines that there is no bubble or air in the protrusion 1402, then the sensor 1408 may communicate with the controller 130 that the protrusion 1402 is full of fluid. The controller 130 may receive data from each sensor 1408 and may then calculate the amount of fluid in the canister 115. In other embodiments, each sensor 1408 of the plurality of sensors 1408 may be an optical sensor or a fiber optic sensor. In embodiments where each sensor 1408 of the plurality of sensors 1408 is an optical sensor or a fiber optic sensor, the plurality of recesses 1406 and the plurality of protrusions 1402 may be transparent to allow the optical sensors or the fiber optic sensors to determine the parameter associated with the canister. The optical sensors or the fiber optic sensors may be able to determine both the amount of fluid in the corresponding protrusion 1402 and the color of the fluid in the protrusion 1402.

In any of the above-described embodiments, the therapy system 100 can be modified to include the airflow device 150 of FIG. 1. The airflow device 150 may be any device that can push or pull air to create a fluid flow such as an axial fan. The airflow device 150 may be configured to generate the fluid flow over the canister 115 when the canister 115 has at least a portion of the canister body 202 or the lid 204 that is liquid-impermeable and vapor permeable. The airflow device 150 may allow the canister 115 to process more fluids than it can physically retain at one time by encouraging evaporation of any water molecules in the fluid. In some embodiments, the airflow device 150 may be located in the therapy unit 160. In other embodiments, the airflow device 150 may be located in the canister 115 or may be outside of both the therapy unit 160 and the canister 115 but may generate the fluid flow towards the liquid-impermeable, vapor-permeable portion of the canister 115. The airflow device 150 may be communicatively coupled to the controller 130 and the controller 130 may actuate the airflow device 150 when the fluid flow would be most optimal to encouraging evaporation. For example, the controller 130 may not actuate the airflow device until the canister 115 is storing a predetermined amount of fluid. In some embodiments, the predetermined amount of fluid may be any amount of fluid in the canister 115. The airflow device 150 may not be actuated by the controller 130 when there is no fluid in the canister 115 to optimize the power consumption of the therapy system 100 and to decrease the sound level of the therapy system 100 to increase patient comfort.

In any of the above-described embodiments, the therapy system 100 can be further modified to include the orientation detector 155 of FIG. 1. The orientation detector 155 may be any device that can be communicatively coupled to the controller 130 and can determine an orientation of the canister 115. In some embodiments, the orientation detector 155 may be a sensor, a gyroscope, an accelerometer, or any other device capable of determining the orientation of the canister 115. The orientation detector 155 may be located within the therapy unit 160, within the canister 115, or outside of either the therapy unit 160 or the canister 115. The orientation detector 155 may communicate the orientation of the canister 115 with the controller 130 and the controller 130 may use that data to determine the amount of fluid in the canister 115. Including the orientation detector 155 may increase the accuracy of the value of the parameter associated with the canister 115 that is calculated by the controller 130. In some embodiments, the orientation detector 155 may be a LSM6DSL MEMS sensor module. The LSM6DSL MEMS sensor module may include a 3D accelerometer and a 3D gyroscope that may communicate the angular position, the tilt direction, the vibration, and other positional data of the canister 115 to the controller 130.

Also provided herein are illustrative example methods for determining the value of the parameter of the canister 115. In some example embodiments, a method for determining the value of the parameter of the canister 115 may include providing the therapy unit 160, the canister 115, and a plurality of sensors such as the sensor array 145. The therapy unit 160 can include at least one wall such as the third wall 244 and the controller 130. The canister 115 can include the canister body 202 and the lid 204. The lid 204 can be configured to attach to the canister body 202 to form the sealed interior of the canister 115. The lid 204 can further be configured to couple to the exterior surface 242 of the third wall 244 of the therapy unit 160. Each sensor of the sensor array 145 can be configured to be communicatively coupled to the controller 130 and to generate a signal indicative of the parameter associated with the canister 115. The method may further include generating the signal indicative of the parameter associated with the canister 115 with each sensor of the sensor array 145, receiving, with the controller 130, the signal from each sensor of the sensor array 145, and determining the value of the parameter associated with the canister in response to the signal from each sensor of the sensor array 145.

In some embodiments, generating the signal with each sensor of the sensor array 145 can include directing a probe of the sensor towards the sealed interior of the canister 115 and sensing fluid in the sensing path of the probe of the sensor. Each sensor may set the signal to a value indicative of a sensed fluid if the sensor senses fluid in the sensing path of the probe or each sensor may set the signal to a value indicative of a lack of fluid if the sensor does not sense fluid in the sensing path of the probe. Each sensor may then send the signal to the controller 130.

In other embodiments, generating the signal with each sensor of the sensor array 145 can include disposing the plurality of sensors 1408 proximate to the interior surface 1410 of the third wall 244 of the therapy unit 160. For each sensor 1408 of the plurality of sensors 1408, the method may include directing a probe of the sensor 1408 towards the corresponding protrusion 1402 of the plurality of protrusions 1402 of the lid 204 of the canister 115. Each protrusion 1402 of the plurality of protrusions 1402 may be configured to be received by the corresponding recess 1406 of the plurality of recesses 1406 of the third wall 244 of the therapy unit 160. The method can further include sensing, with the sensor 1408, fluid in the protrusion 1402. The signal of the sensor 1408 can be set to a value indicative of a sensed fluid if the sensor 1408 senses fluid in the protrusion 1402 and can be set to a value indicative of a lack of fluid if the sensor does not sense fluid in the protrusion 1402. The method may further include sending the signal from the sensor 1408 to the controller 130.

In some embodiments, the method can further include providing the user interface 210. The user interface 210 can be communicatively coupled to the controller 130. The user interface 210 can then receive the value of the parameter associated with the canister 115 from the controller 130 and output the value of the parameter associated with the canister 115.

The method can further include storing historical data associated with the parameter associated with the canister 115 in the controller 130 and determining the value of the parameter associated with the canister 115 in response to the historical data associated with the parameter associated with the canister 115. In some embodiments, the method can further include providing the orientation detector 155 configured to generate an orientation signal indicative of the orientation of the canister 115. The orientation detector 155 can be configured to be communicatively coupled to the controller 130. The method can further include receiving the orientation signal with the controller 130 from the orientation detector 155 and determining the value of the parameter associated with the canister 115 in response to the orientation signal.

The systems, apparatuses, and methods described herein may provide significant advantages. For example, the therapy system 100 may allow for improved fill level readings of the canister 115. In some embodiments, the canister 115 may be opaque to provide an improved user experience by obscuring any stored fluid in the canister 115 from view of the user. Improved fill level readings may allow for fewer changes of the canister 115 and for a reduction in false alarms indicating that the canister 115 is full. The therapy system 100 may also allow for improved evaporative technologies which may improve efficiency and performance of the therapy system 100. The use of the sensor array 145 may allow for remote monitoring of the canister 115 and of the therapy system 100 which may further improve the user experience.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the canister 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A system for detecting a value of a parameter of a canister, the system comprising:
a therapy unit comprising:
at least one wall having an interior surface and an exterior surface; and
a controller;
a canister comprising:
a canister body; and
a lid configured to be coupled to the canister body to form a sealed interior, the lid further configured to be coupled to the exterior surface of the at least one wall of the therapy unit; and
at least one sensor configured to be communicatively coupled to the controller and to generate a signal indicative of a parameter associated with the canister, the controller being configured to receive the signal from the at least one sensor and to determine a value of the parameter.

2. The system of claim 1, wherein the parameter is an amount of fluid in the sealed interior of the canister.

3. The system of claim 1, wherein the system further comprises a user interface configured to be communicatively coupled to the controller and to output the value of the parameter associated with the canister.

4. The system of claim 1, wherein the at least one sensor comprises a sensor array.

5. The system of claim 1, wherein the at least one sensor is coupled to the interior surface of the at least one wall and a probe of the at least one sensor is directed towards the exterior surface of the at least one wall.

6. The system of claim 5, wherein:
the at least one wall of the therapy unit comprises at least one cavity, the at least one sensor disposed proximate to the at least one cavity;
the lid comprises at least one cavity; and
the at least one cavity of the lid is configured to align with the at least one cavity of the at least one wall of the therapy unit.

7-8. (canceled)

9. The system of claim 1, wherein the at least one sensor comprises a non-contact fluid sensor.

10. The system of claim 1, wherein the at least one sensor is integrated into at least one of the lid and the canister body of the canister.

11. The system of claim 1, wherein
the lid of the canister comprises at least one protrusion configured to align with at least one recess on the at least one wall of the therapy unit;
the at least one sensor is proximate to the interior surface of the at least one wall of the therapy unit and a probe of the at least one sensor is directed towards the at least one recess on the at least one wall of the therapy unit; and
the parameter comprises an amount of fluid in the at least one protrusion of the lid of the canister.

12. (canceled)

13. The system of claim 11, wherein the at least one sensor is an air bubble fluid sensor.

14. The system of claim 11, wherein the at least one sensor is an optical sensor and the parameter is a color of fluid in the sealed interior of the canister.

15. (canceled)

16. The system of claim 1, further comprising an orientation detector configured to generate a signal indicative of an orientation of the canister, the orientation detector configured to be communicatively coupled to the controller, wherein the controller is further configured to receive the signal from the orientation detector to determine the value of the parameter associated with the canister.

17. (canceled)

18. The system of claim 1, wherein the therapy unit further comprises an airflow device, the airflow device configured to be actuated by the controller in response to the parameter associated with the canister.

19. A method of determining a value of a parameter of a canister, the method comprising:
providing a therapy unit comprising:
at least one wall having an interior surface and an exterior surface; and
a controller;
providing a canister comprising:
a canister body; and
a lid configured to attach to the canister body to form a sealed interior, the lid further configured to couple to the exterior surface of the at least one wall of the therapy unit;
providing a plurality of sensors, each sensor of the plurality of sensors configured to be communicatively coupled to the controller and to generate a signal indicative of a parameter associated with the canister;
generating a signal indicative of the parameter associated with the canister with each sensor of the plurality of sensors;
receiving, with the controller, the signal from each sensor of the plurality of sensors; and
determining a value of the parameter associated with the canister in response to the signal from each sensor of the plurality of sensors.

20. The method of claim 19, further comprising:
providing a user interface communicatively coupled to the controller;
receiving, with the user interface, the value of the parameter associated with the canister from the controller; and
outputting, with the user interface, the value of the parameter associated with the canister.

21. (canceled)

22. The method of claim 19, wherein the parameter is an amount of fluid in the sealed interior of the canister and generating the signal with each sensor of the plurality of sensors comprises:
for each sensor of the plurality of sensors:
directing a probe of the sensor towards the sealed interior of the canister;
sensing, with the sensor, fluid in a sensing path of the probe of the sensor;
setting the signal to a value indicative of a sensed fluid if the sensor senses fluid in the sensing path of the probe;
setting the signal to a value indicative of a lack of fluid if the sensor does not sense fluid in the sensing path of the probe; and
sending the signal from the sensor to the controller.

23. The method of claim 19, wherein the parameter is an amount of fluid in the sealed interior of the canister and generating the signal with each sensor of the plurality of sensors comprises:
disposing the plurality of sensors proximate to the interior surface of the at least one wall of the therapy unit;
for each sensor of the plurality of sensors:
directing a probe of the sensor towards a protrusion of a plurality of protrusions of the lid of the canister, each protrusion of the plurality of protrusions configured to be received by a recess of a plurality of recesses of the at least one wall of the therapy unit;
sensing, with the sensor, fluid in the protrusion;
setting the signal of the sensor to a value indicative of a sensed fluid if the sensor senses fluid in the protrusion; and
setting the signal of the sensor to a value indicative of a lack of fluid if the sensor does not sense fluid in the protrusion; and
sending the signal from the sensor to the controller.

24.-25. (canceled)

26. A canister comprising:
a canister body;
a lid configured to attach to the canister body to form a sealed interior; and
at least one sensor comprising a probe directed towards the sealed interior of the canister, the at least one sensor configured to generate a signal indicative of a parameter associated with the canister.

27. (canceled)

28. The canister of claim 26, wherein the canister body is opaque, the lid is opaque, and the lid includes a window configured to expose a portion of the sealed interior of the canister.

29. The canister of claim 26, wherein the lid is transparent.

30.-39. (canceled)
